(19) United States
(12) Patent Application Publication
(10) Pub. No.: US 2009/0053290 A1
(43) Pub. Date: Feb. 26, 2009

(54) TRANSDERMAL DRUG DELIVERY COMPOSITIONS AND TOPICAL COMPOSITIONS FOR APPLICATION ON THE SKIN

(76) Inventors: Bruce J. Sand, Westlake Village, CA (US); Michael Babich, Westlake Village, CA (US); Ali Zendedel Haghighi, Westlake Village, CA (US)

Correspondence Address: CHRISTIE, PARKER & HALE, LLP, PO BOX 7068, PASADENA, CA, 91109-7068, US

(21) Appl. No.: 12/281,768

(22) PCT Filed: Mar. 8, 2007
(86) PCT No.: PCT/US07/06037

§ 371 Date: Oct. 15, 2008

Related U.S. Application Data
(60) Provisional application No. 60/878,886, filed on Jan. 3, 2007.
(60) Provisional application No. 60/796,007, filed on Apr. 28, 2006.
(60) Provisional application No. 60/781,951, filed on Mar. 8, 2006.
(60) Provisional application No. 60/781,950, filed on Mar. 8, 2006.
(60) Provisional application No. 60/781,925, filed on Mar. 8, 2006.
(60) Provisional application No. 60/801,349, filed on May 18, 2006.
(60) Provisional application No. 60/781,952, filed on Mar. 8, 2006.

(51) Int. Cl. A61K 9/70; A61K 47/42; A61K 47/10; A61K 47/02; A61K 31/4172; A61K 31/351; A61K 39/08; A61K 9/10; A61K 31/496; A61K 31/164; A61K 31/4545; A61K 31/7042; A61K 31/60

(52) U.S. Cl. 424/449; 514/773; 514/772; 514/769; 514/400; 514/459; 424/236.1; 514/254.07; 514/626; 514/290; 514/37; 514/159; 424/484

(57) ABSTRACT

Transdermal delivery compositions and topical compositions for application to the skin are provided. The transdermal delivery composition includes at least two penetrants working synergistically but by disparate biochemical pathways. In one embodiment, the transdermal delivery system includes benzyl alcohol and lecithin organogel. The transdermal delivery compositions are used in a variety of topical compositions as a means of transdermally delivering and topically administering different drugs and agents, including compositions promoting collagen biosynthesis, retinoids and skin lighteners, chemical denervation agents such as BOTOX®, anti-fungal agents, anesthetics and non-steroidal anti-inflammatory drugs (NSAIDs). In addition, these topical compositions may be used in combination with non-ablative treatment modalities, such as microdermabrasion, laser-based skin remodeling and radio-frequency-based skin remodeling.

### FIELD OF THE INVENTION

The invention is directed to transdermal drug delivery compositions and to topical compositions for application on the skin.

### BACKGROUND OF THE INVENTION

The skin can develop a host of maladies but is impermeable to most agents, posing a challenge to the topical treatment of most maladies. To be effective, the active drug or agent in a topical composition must penetrate the skin, which is a structurally complex and relatively thick membrane. Molecules moving through the skin must first penetrate the stratum corneum and any material on its surface. The molecules must then penetrate the viable epidermis, the papillary dermis, and the capillary walls into the vascular system or lymphatic system. To be absorbed, the molecules must overcome a different resistance to penetration in each type of tissue. This makes transport across the skin a complex procedure. The cells of the stratum corneum present the primary barrier to absorption of topical compositions or the trans-epidermal administration of drugs or medicaments. The stratum corneum is a thin layer of dense, highly keratinized cells approximately 10-15 microns thick, and covers most of the human body. The high keratinization of these cells and their dense packing creates, in most cases, a barrier substantially impermeable to drug penetration. Many drugs permeate through the skin very slowly. Some metabolic interventions may help enhance permeation. Most of these metabolic interventions create phase separation in the membrane. The formation of non-lamellar domains leads to additional potential pathways for transdermal drug delivery.

Strategies have been devised to enhance transdermal drug delivery, and these strategies can be categorized as either physical, chemical, mechanical or biochemical. Combinations of these strategies may also be used to increase efficacy or to extend the time for transdermal delivery.

Physical techniques vary from straightforward approaches, such as occlusion and tape stripping, to the use of highly sophisticated instrumentation and miniaturization (e.g. iontophoresis and electroporation). One of the most straightforward physical methods is prolonged occlusion, which alters the barrier properties of the stratum corneum. After 24 to 28 hours of occlusion with resultant hydration, corneocytes swell, intercellular spaces become distended, and the lacunar network becomes dilated. Distention of the lacunae eventually leads to connections with an otherwise discontinuous system, creating pores in the stratum corneum interstices through which polar and non-polar substances can penetrate more readily.

Stripping is another straightforward physical method to abrogate the barrier. Sequential stripping, with either adhesive tapes or cyanoacrylate glue, increases transepidermal water loss (“TEWL”), an indicator of barrier defects. This correlates with enhanced transdermal drug delivery. Tape stripping removes both corneocytes and extracellular lipids, thereby reducing the elongated path that drugs otherwise need to traverse. In addition, tape stripping mechanically disrupts lamellar bilayers, even in retained, lower stratum corneum layers. However, to effectively disrupt the barrier by such a process, multiple strippings are required. Such multiple strippings can result in mast cell degranulation and inflammation, leading to discomfort as well as post-inflammatory hyperpigmentation. Also, even more strippings may be necessary to disrupt the barrier in lightly pigmented subjects.

Iontophoresis and electroporation are electrically assisted, physical methods of enhancing delivery of drugs or macromolecules across the stratum corneum. Iontophoresis uses low currents from an externally placed electrode (having the same charge as the net polarity of the drug) to drive the molecules across the stratum corneum. Although the predominant pathway of iontophoretic transport is appendegeal (i.e. through the hair follicles and/or sweat glands), extracellular routes are also traversed. Iontophoretic delivery through the stratum corneum interstices occurs via aqueous pores, thereby operating at both a macro (appendegeal) and micro (extracellular and lacunar) level. Because drug delivery is proportionate to the amount of applied current, iontophoresis allows for programmable drug delivery, which can be accomplished more easily due to recent developments in miniaturized microprocessor systems and disposable hydrogel pads.

Electroporation is a relatively new non-thermal, electrical method. It employs ultra-short pulses with large trans-membrane voltages to induce structural rearrangement and conductance changes in membranes, leading to pore formation. Though it is most effective for single bilayer membranes, electroporation also permeabilizes the human stratum corneum. Although pore formation is largely considered to be the subcellular mechanism, the actual pathway across the stratum corneum is not yet known.

Ultrasound and sonophoresis are other methods for permeabilizing the stratum corneum. These methods are extensively employed in both medical diagnosis and physical therapy and are widely considered safe with no known short or long term side effects. According to these methods, when ultrasound waves encounter the stratum corneum they generate defects in the structure which permeabilize the stratum corneum. Lower frequencies ranging from 1-3 MHz are minimally effective, but higher frequencies ranging from 10-20 MHz significantly enhance drug delivery across the stratum corneum.

During sonophoresis, electron-dense tracers, such as lanthanum and FITC-conjugated dextran, penetrate across the stratum corneum into the epidermis and dermis within five minutes with no apparent damage to keratinocytes. Moreover, tracer movement occurs through the lacunae, which become dilated and transiently continuous. Thereafter, the pore pathway collapses upon cessation of the applied energy.

Another recently developed technique utilizes laser beams to generate photomechanical stress waves that interact directly with the stratum corneum in ways different from ultrasound waves. These stress waves are generated by ablation of a target material that covers the drug-containing solution to be delivered. The target first absorbs the laser radiation, and the solution then serves as a coupling medium for the stress waves to propagate the drug across the stratum corneum. As in sonophoresis and iontophoresis, the permeation pathway is believed to be extracellular, but the actual pathway is not yet known. Also, as in sonophoresis and iontophoresis, single photomechanical compression waves modulate the permeability of the stratum corneum only transiently, and the barrier function recovers almost immediately.

Chemical methods of enhancing transdermal drug delivery are more commonly used and include the use of chemical enhancers to increase permeability of the stratum corneum. Chemical enhancers are compounds delivered along with the intended drug, or prior to drug administration, and have been used to increase the rate at which drugs penetrate the skin. Ideally, such chemical enhancers are passive and innocuous and merely facilitate diffusion of the intended drug through the stratum corneum. Although the permeability of many therapeutic agents may be increased using these chemical enhancers, high levels of certain enhancers may result in skin irritation and sensitization problems.

Solvents, such as ethanol, methanol, chloroform and acetone, as well as detergents, can extract stratum corneum barrier lipids and permeabilize the stratum corneum. Morphological changes in the human stratum corneum following extensive exposure to such solvents include phase separation and derangement of lamellar bilayers in addition to the creation of defects in corneocytes. Surfactants, such as sodium dodecyl (lauryl) sulfate (SDS), and vehicles (e.g. propylene glycol) extract lipids, and create extensive expansion of pre-existing lacunar domains. Moreover, solvent-based penetration enhancers, such as azone, sulfoxides, urea and FFA, not only extract extracellular lipids, but also alter the stratum corneum lipid organization (phase behavior), thereby enhancing transdermal delivery and expanding intercellular domains.

Liposomes are another chemical method of permeabilizing the stratum corneum and are frequently used to enhance drug delivery. However, liposomes appear to enhance transdermal drug delivery solely by the appendegeal pathway, and it is not yet known whether they penetrate the intact stratum corneum.

There are many known chemical permeation enhancers. However, these known chemical permeation enhancers are only minimally effective in increasing the rate at which drugs permeate the skin. In addition, the known chemical permeation enhancers may cause skin damage, irritation, sensitization, or the like, and cannot be used to effect transdermal delivery of high molecular weight drugs such as peptides, proteins and nucleic acids.

Although a wide variety of methods have been used to enhance drug delivery, as discussed above, these methods are only minimally effective. A major problem with these methods is that they are assessed in vitro, using devitalized human skin. Non-viable skin samples do not mount a metabolic response against barrier perturbations, and such in vivo repair responses inevitably restrict the efficacy of any enhancement method (i.e. they “close the window”).

Accordingly, an alternative approach is to enhance the efficacy of standard enhancers by inhibiting the repair (metabolic) response in vivo. Such a metabolic approach could be used in conjunction with another method to further increase efficacy. Some of these methods can abrogate the barrier of intact skin by “opening the window,” thereby obviating the requirement for pre-treatment or co-treatment with a primary enhancer.

Such a biochemical approach to enhance transdermal drug delivery has led to the development of another category of enhancers, i.e. biochemical enhancers. These biochemical enhancers alter the supramolecular organization of preformed lamellar bilayers. These enhancers include: (1) synthetic analogues of Chol, Cer and FFA, such as trans-vaccenic acid and epicholesterol, which induce abnormalities in lamellar membrane organization; (2) complex precursors of Chol, Cer and FFA, such as sterol esters, which are not efficiently metabolized to their respective products in the stratum corneum, thereby providing non-lamellar phase separation; (3) supraphysiologic concentrations of physiologic lipids, such as Chol sulfate, which can also induce phase separation in preformed membrane bilayers; and (4) hydrolytic enzymes, such as acid ceramidase, which degrade one or more of the three key stratum corneum species. The result of phase separation is a more permeable stratum corneum interstices, due not only to deletion of key hydrophobic lipids, but also to the creation of additional penetration pathways, distinct from the primary, lamellar membrane route.

Other factors affecting transdermal transport of drugs include those involved in the pharmacokinetics of the skin. Four factors control the kinetics of percutaneous absorption of drugs across the skin barrier. The first factor is the bioavailability of the drug, which is determined by the drug vehicle and affected by the link between the drug's potency and therapeutic effectiveness. The second factor is the concentration of the soluble drug in the drug vehicle. This is the driving force for percutaneous absorption. The third factor is the partition coefficient. Topically applied drugs are poorly absorbed generally because only a small fraction of the drug partitions into the stratum corneum. The fourth factor is the regional variation, such as the thickness of the thickness of the stratum corneum. Such variations will modulate drug absorption.

### SUMMARY OF THE INVENTION

The present invention is directed to the transdermal delivery of a variety of drugs and compositions. In one embodiment of the present invention, in fact, a transdermal delivery composition is provided that includes at least two penetrants working synergistically but by disparate biochemical pathways. In an exemplary embodiment, the transdermal delivery composition includes both benzyl alcohol and lecithin organogel. These two penetrants provide a particularly effective means of transdermally delivering a wide variety of payloads through the epidermis and stratum corneum. In addition, this effective means of transdermal transport of drugs, agents and compositions makes the delivered agent more bioavailable in smaller doses and increases bio activity. This, in turn, reduces the side effects normally associated with the target drug or agent and reduces systemic toxicity.

According to an alternative embodiment of the present invention, topical compositions a and methods are provided for the topical application of compositions or the promotion of collagen biosynthesis. One exemplary composition includes methionine and cysteine; a mixture of other amino acids including leucine, lysine, phenylalanine, threonine, tryptophan, valine, histidine and arginine; a least one antioxidant; at least one cross-linking agent; at least one metallic catalyst; at least one penetrant or transdermal delivery agent or composition and a topical pharmaceutically acceptable carrier.

In an alternative embodiment, topical compositions and methods are provided for the topical application of retinoids and/or skin lighteners. An exemplary composition includes a skin lightener selected from hydroquinone, a hydroquinone derivative, kojic acid, azelaic acid, glycolic acid and artocarpin; a skin penetrant or transdermal delivery agent or composition; and a topical pharmaceutically acceptable carrier. In another exemplary embodiment, the composition includes a retinoid in place of or in addition to the skin lightener.

In yet another embodiment of the invention, topical compositions and methods are provided for the topical application of chemical denervation agents, such as botulinium toxins. One exemplary composition includes a chemodenervation agent, a permeation enhancer or transdermal delivery agent or composition, and a topical pharmaceutically acceptable carrier.

According to still another embodiment of the invention, topical compositions and methods are provided for the topical application of anti-fungal agents. An exemplary composition includes an anti-fungal agent, a permeation enhancer or transdermal delivery agent or composition and a topical pharmaceutically acceptable carrier. Exemplary anti-fungal agents include fungicidal and fungistatic agents including terbinafine, itraconazole, micronazole nitrate, thiapendazole, tolnaftate, clotrimazole and griseofulvin.

In still yet another embodiment of the present invention, topical compositions and methods are provided for the topical application of anesthestics. An exemplary composition includes at least one anesthetic, a permeation enhancer or transdermal delivery agent or composition and a topical pharmaceutically acceptable carrier. Non-limiting examples of suitable anesthetics include benzocaine, lidocaine, tetracaine, bupivacaine, cocaine, etidocaine, mepivacaine, pramoxine, prilocalne, procaine, cnloroprocaine, oxyprocaine, proparacaine, ropivacaine, dyclonine, dibucaine, propoxycaine, chloroxylenol, cinchocaine, dexivacaine, diamocaine, hexylcaine, levobupivacaine, propoxycaine, pyrrocaine, risocaine, rodocaine, and pharmaceutically acceptable derivatives and bioisosteres thereof. In one embodiment, the at least one anesthetic includes benzocaine, lidocaine and tetracaine.

In another embodiment of the invention, topical compositions and methods are provided for the topical application of non-steroidal anti-inflammatory drugs (NSAIDs). An exemplary composition includes an NSAID, a permeation enhancer or transdermal delivery agent or composition and topical pharmaceutically acceptable carrier. Non-limiting examples of suitable NSAIDs include aspirin, salsalate, diflunisal, ibuprofen, ketoprofen, nabumetone, piroxicam, naproxen, diclofenac, indomethacin, sulindac, tolmetin, etodolac, detorolac, oxaprozin, celecoxib and pharmaceutically acceptable derivatives thereof. A single NSAID may be used, or alternatively, a combination of NSAIDs may be used.

### BRIEF DESCRIPTION OF THE DRAWINGS

These and other features and advantages of the present invention will be better understood by reference to the following detailed description when considered in conjunction with the accompanying drawings in which:

FIG. 1 is a graph of the concentration mass of iron (Fe) in samples collected at four different time points;

FIG. 2 is a graph of the concentration mass of copper (Cu) in samples collected at four different time points;

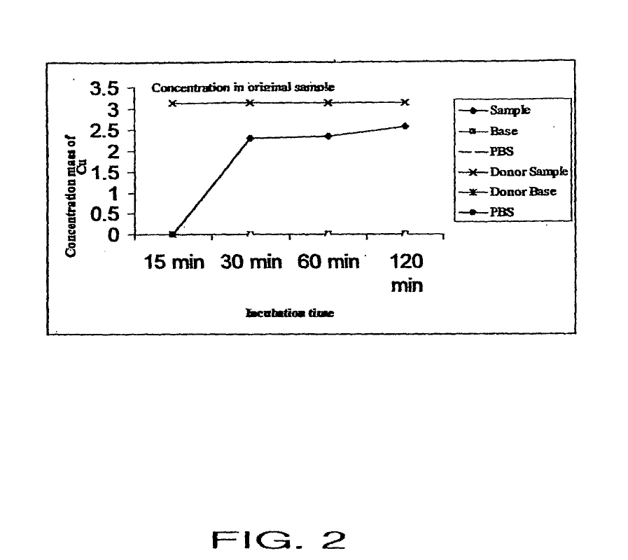

FIG. 3 is a graph of amplification plot data using pro-collagen primers and probes;

FIG. 4 is a graph depicting the theoretical advantages of transdermal delivery, which include less toxicity and improved efficacy;

FIG. 5 is a graph showing the lack of toxicity of proanthocyanidin evaluated using human skin fibroblasts grown in 10% FBS/DMEM;

FIG. 6 is a graph showing the cytotoxicity of glutaldehyde evaluated using human skin fibroblasts grown in 10% FBS/DMEM;

FIG. 7 is a graph showing the relationship between cross-linking effectiveness (judged by melting temperature) and proanthocyanidin concentration;

FIG. 8 is a graph showing the results of collagenase digestion of proanthocyanidin-treated collagen sponges and controls (open bar, untreated control; shaded bar, treatment with proanthocyanidin);

FIG. 9 is a graph showing the effect of proanthocyanidin on cell proliferation and synthesis of collagen in vitro using human skin fibroblasts cultured on proanthocyanidin-treated ornon-treated pericardium tissue (untreated, open bars; proanthocyanidin-treated, shaded bars);

FIG. 10 is a graph showing the changes in the shrinkage temperature of tissues stored in two different solutions (a) PBS (solid line) and (b) 40% ethanol/PBS (dashed line); and

FIG. 11 depicts the chemical structure of monomer (A) and dimer (B) forms of proanthocyanidin.

### DETAILED DESCRIPTION OF THE INVENTION

Certain embodiments of the present invention are directed to topical compositions for the treatment of skin ailments. According to some exemplary embodiments, the topical compositions can be used alone to treat the specified skin ailment. In alternative embodiments, the topical compositions can be used in a multi-phasic treatment combining the composition with certain other methods for increasing the permeability of the skin to the topical compositions. Whether topical compositions are used alone or in combination with other methods for increasing skin permeability, the results of the treatment are natural, rapid, and long-lasting.

### I. Transdermal Delivery

The inventive topical compositions and methods can be used to treat a wide variety of skin ailments. The topical composition includes a transdermal drug delivery composition which carries a target drug through the outer-most layer of the skin (the epidermis), delivering the drug to the inner layer of the skin (the dermis) to effect treatment of the specified ailment. This penetration of the epidermis is essential to make the target drug bioavailable to the dermis.

The skin is the most extensive and readily accessible organ of the human body, but it presents a formidable barrier preventing penetration of most substances through its surface. The outer-most layer of skin (the epidermis) forms a relatively thin coating, which serves as a barrier between the skin and the environment. The most superficial area of the epidermis (the stratum corneum (SC)) serves as a protective impediment. The rate-limiting step in the absorption of most agents through the skin appears to be passage through the stratum corneum.

The barrier to penetration provided by the stratum corneum gives the skin a low permeability to most agents. This low permeability necessitates the use of penetration enhancers to increase the skin's permeability. One major difficulty associated with the use of penetration enhancers is lack of specificity. This is true for drugs as well as cosmetic skin care formulations designed to rejuvenate aged or otherwise damaged skin. Despite efforts to enhance transdermal drug delivery, the list of drugs capable of transdermal delivery is quite small, and generally limited to lipophilic compounds of both low molecular weight and low total absorbed dose.

Transdermal transport of drugs (i.e., the transport of pharmacologically active compounds through the skin) is an important alternative to the classical methods of drug delivery. The chemical structure needed to penetrate through the skin is not yet well understood, but even if it were, chemically modifying all drugs of interest to make them transdermally active is not practical. Accordingly, certain embodiments of the present invention are directed to transdermal drug delivery compositions that interact with the skin to allow various molecules to pass to the inner layers of the skin. This can be accomplished by the degradation of corneodesmosomes to form discontinuous lacunar domains, which represent the likely aqueous ‘pore’ pathway. These lacunae can enlarge and extend, forming a continuous, but collapsible network under certain conditions, e.g. prolonged hydration, sonophoresis.

These transdermal delivery compositions can be combined with the target drug into a single topical composition for the treatment of a specified skin ailment. Alternatively, the skin can be treated with an inventive transdermal delivery composition prior to the topical administration of the target drug. Treating the skin with a transdermal delivery composition helps permeabilize the stratum corneum and epidermis to enhance the passage of the target drug that is later topically administered.

One embodiment of a transdermal drug delivery composition includes two or more transdermal penetrants working synergistically but by disparate biochemical pathways. As used herein, the term “penetrant” refers to agents or compounds capable of penetrating the outer layers of the skin and/or agents or compounds capable of enhancing the permeability of the skin. These inventive transdermal delivery compositions transport target drugs or agents through the epidermis rapidly into the dermis. According to one embodiment, the delivery composition includes a first penetrant and a second penetrant, where each of the first and second penetrants is any suitable agent capable of penetrating the stratum corneum. The first and second penetrants work synergistically to enhance permeability of the outer skin layers and may follow disparate biochemical pathways.

Non-limiting examples of suitable penetrants for use as the first and second penetrants include lower alkyl diols, C₁₀-C₂₀ fatty acids and esters, C₄-C₂₀ unsubstituted aliphatic alcohols, and C₄-C₂₀ substituted aliphatic alcohols. Other non-limiting examples of suitable penetrants include dimethyl sulfoxide, N,N-dimethyl acetamide, 2-pyrrolidone, 1-methyl-2-pyrrolidone, carbitol solvent (available from Union Carbide), propylene carbonate, 1,5-dimethyl-2-pyrrolidone, and 2-pyrrolidone-5-carboxylic acid.

Still other non-limiting examples of suitable penetrants include mixtures of 1-dodecylazacycloheptan-2-one with a diol compound or a second N-substituted alkyl-azacycloalkyl-2-one (“cycloketo” compound). Non-limiting examples of suitable diol compounds for use in such mixtures include 1,2-propanediol, 1,3-propanediol, 1,2-butanediol, 1,3-butanediol, 1,4-butanediol, and 2,3-butanediol. Non-limiting examples of suitable “cycloketo” compounds include those represented by Formula 1, below.

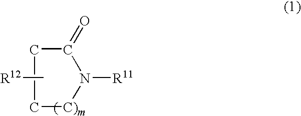

In Formula 1, R¹¹ is selected from —H, —CH₃, —C₂H₅, —C₂H₄OH, —C₃H₇, —C₃H₆OH, and CH₂CHOHCH₂OH. R¹² is selected from —H, —CH₃, —C₂H₅, —C₃H₇, and —C₄H₉, and m is an integer ranging from 0 to 2.

Yet other non-limiting examples of suitable penetrants for use as the first and second penetrants include aminopolysaccharides such as chitosonium polymers and covalent derivatives of chitosan prepared by the reaction of chitosan with one or more electrophilic reagents such as ethylene oxide, propylene oxide, glycidol, C₁-C₂₄ alkyl halides, glycidyl C₁-C₂₄ trialkylammonium salts, 3-chloro-2-hydroxypropyl ammonium salts, 1,3-propanesultone, haloacetates, succinic anhydride, maleic anhydride, carboxylic acyl halides, N-carboxy-α-carboxylic acyl halides, N-carboxy-α-amino acid anhydrides, and other electrophilic reagents.

Still other non-limiting examples of suitable penetrants include diisopropyl adipate, dimethyl isosorbide, propylene glycol, and 1,2,6-hexanetriol. More non-limiting examples of suitable penetrants include dioctyl maleate, propylene carbonate, and diisopropyl sebacate. Even more non-limiting examples of suitable penetrants include dual phase solvent carrier systems of benzyl alcohol and a fugitive solvent having a boiling point of less than about 110° C.

Specific, non-limiting examples of suitable penetrants include sulfoxides such as dimethylsulfoxide (DMSO) and decylmethylsulfoxide (C₁₀MSO); ethers such as diethylene glycol monoethyl ether (available commercially as Transcutol™) and diethylene glycol monomethyl ether; surfactants such as sodium laurate, sodium lauryl sulfate, cetyltrimethylammonium bromide, benzalkonium chloride, Poloxamer (231, 182, 184), Tween (20, 40, 60, 80), and lecithin; the 1-substituted azacycloheptan-2-ones including 1-n-dodecylcyclazacycloheptan-2-one (available commercially as Azone™); alcohols such as ethanol, propanol, octanol, and the like; fatty acids such as lauric acid, oleic acid and valeric acid; fatty acid esters such as isopropyl myristate, isopropyl palmitate, methyl propionate, and ethyl oleate; polyols and esters thereof such as propylene glycol, ethylene glycol, glycerol, butanediol, polyethylene glycol, and polyethylene glycol monolaurate (PEGML); amides and other nitrogenous compounds such as urea, dimethylacetamide (DMA), dimethylformamide (DMF), 2-pyrrolidone, 1-methyl-2pyrrolidone, ethanolamine, diethanolamine and triethanolamine; terpenes; alkanones; and organic acids such as salicylic acid and salicylates, citric acid, and succinic acid.

In one exemplary embodiment, the first penetrant is an aliphatic alcohol substituted with an aromatic substituent. Non-limiting examples of suitable such alcohols include benzyl alcohol and phenethyl alcohol. In one embodiment, for example, the first penetrant is benzyl alcohol. Benzyl alcohol acts by basically dissolving and removing the biphasic layer of the cell membrane. This removes the barrier, resulting in the rapid transport of the target agent or composition across the cell membrane.

The first penetrant (e.g. benzyl alcohol) may be present in the transdermal delivery composition in an amount ranging from about 1% to about 20% by weight. According to another embodiment, the first penetrant is present in an amount ranging from about 5% to about 15% by weight. In still another embodiment, the first penetrant is present in an amount ranging from about 1.5% to about 2.5% by weight. In yet another embodiment, the first penetrant is present in an amount of about 10% by weight. In still, yet another embodiment, the first penetrant is present in an amount of about 2% by weight.

The second penetrant may be any suitable penetrant capable of working synergistically with the first penetrant. The second penetrant may also be any penetrant following a biochemical pathway disparate from the pathway followed by the first penetrant. The second penetrant is present in the delivery composition in an amount ranging from about 0.5% to about 20% by weight. In one exemplary embodiment, the second penetrant is present in an amount ranging from about 0.6 to about 20% by weight. In an alternative embodiment, the second penetration is present in an amount ranging from about 0.5 to about 15% by weight. In another alternative embodiment, the second penetrant is present in an amount of about 0.6% by weight. In still, yet another embodiment, the second penetrant is present in an amount of about 0.5% by weight.

In one embodiment, for example, the second penetrant is a lecithin organogel. The lecithin organogel may include soybean lecithin (Epicuron 200) containing at least about 95% phosphatidylcholine. The solvent may be any suitable solvent. One exemplary biocompatible solvent is isopropyl palmitate.

Lecithin organogels are suitable for cosmetic and pharmacological applications. Water can be used as a gel inducer and can be substituted for other substances, such as glycerol or other low molecular weight, hydrogen bonding liquids.

Lecithin organogels are particularly useful as they are capable of hosting various guest molecules. For example, lipophilic, hydrophilic and amphoteric molecules, including enzymes, can be solubilized in the gels. The biocompatibility and ability of lecithin organogels to solubilize drugs makes them a good matrix for transdermal transport. Lecithin gels kept at constant temperature are indefinitely stable in closed vials, without changing in color or appearance. Even gels in open vials stored at room temperature remain stable for at least 30 days. The gels do not absorb significant amounts of humidity from the air during storage. The same is true for gels containing solubilized guest molecules, such as vitamin A palmitate. In addition, lecithin organogels can be prepared easily and rapidly and are biocompatible. They are transparent and remain stable for long periods of time. They can carry sizeable amounts of very different chemicals as guest molecules, such as amino acids and peptides, and have great potential for fast transdermal transport. These gels are not harmful to the skin. In particular, the stratum corneum remains intact after prolonged contact with the gels.

It is believed that lecithin organogels effect transport by slightly disorganizing the structure of the skin, thus permitting permeation of various substances. The stratum corneum contains regularly arranged layers of lipids such that the transport mechanism depends on the interaction between the lipids and the phospholipids of the gel.

In one embodiment, the second penetrant is pluronic lecithin organogel (“PLO”), which combines a lecithin organogel with a surfactant (i.e. Pluronic 127). PLO is a microemulsion having reversed polymer-like micelles. PLO can be used as a vehicle for anti-inflammatory drugs or pain relievers. PLO dissolves and incorporates into the biphasic layer of the cell membrane, thus transporting the target agent or composition through the membrane. PLO can transfer compounds at a much higher concentration and a lower lever of dispersion. When PLO is the second penetrant, it may be present in the transdermal delivery composition in an amount ranging from about 0.5 to about 15% by weight.

The action of benzyl alcohol is different than that of pluronic lecithin organogel (PLO) with regard to the transfer of compounds across a membrane such as skin. Benzyl alcohol can dissolve the bilayer membrane of the skin by dissolving the lipid portion of the structure. By doing so, the drug or compound dissolved in the benzyl alcohol has better access to the inner layers of the skin. Also, due to its bipolar nature, benzyl alcohol effects transdermal delivery better than other alcohols, such as methanol and ethanol. Due to the aromatic group (i.e. benzene) in benzyl alcohol the molecule has a polar (at the alcohol end) and a non-polar end (the benzene end). This enables benzyl alcohol to dissolve a wider variety of drugs (which are generally non-polar) and carry them to the inner layers of skin by the lipid dissolving action of the alcohol end.

Lecithin organogels are also bipolar molecules. However, its transdermal delivery action is different that that of benzyl alcohol. the intended drug molecule is present in the micelle of the lecithin organogel. The micelle is such that the non-polar end is toward the center and the polar end is toward the outside. The interaction between the lipid layer of the skin and the polar end of the lecithin organogel (the phospholipid groups) makes it possible for the lecithin organogel to enter the skin layers. Lecithin organogels effect transdermal delivery better than other organic solvents because lecithin organogels can dissolve a wider range of drugs molecules and can deliver the drug molecules to the intended site under the skin at much higher concentrations. This is because there is very little diffusion of the drug molecule as it penetrates through the skin.

In one embodiment of the present invention, as discussed above, a combination of benzyl alcohol and lecithin organogel are used for transdermal delivery of drug compounds. Such a combination of transdermal delivery agents not only takes advantage of the ability of benzyl alcohol to dissolve the lipid layers and increase the speed of access to the lower layers of the skin, but also effects delivery of much higher concentrations of the target drug by action of the lecithin organogel. One exemplary, multi-phasic use of such a combination includes dissolving the drug molecule in lecithin organogel, but first applying the benzyl alcohol to the skin followed by application of the lecithin organogel containing the drug.

According to another embodiment, the transdermal delivery composition may further include two or more metallic cations as enzymatic co-factors. Transdermal delivery compositions according to this embodiment include the epidermal penetrants and the metal cations in a pharmaceutically acceptable carrier to ensure bioavailability. Drugs delivered by these delivery compositions will remain bioactive in the dermis. The use of metal cations in the inventive transdermal delivery compositions enables delivery of bioavailable formulations directly to the targeted extracellular matrices within the dermis without the need for substrates, such as amino acid substrates. In one exemplary embodiment, the transdermal delivery composition includes about 2% by weight benzyl alcohol, from about 0.6 to about 20% by weight of a lecithin organogel and two or more metal cation (such as Fe or Ca) peptides.

Metal cations function as catalysts in several natural biochemical processes, including collagenesis and cell proliferation. In particular, metal cations act as catalysts in several processes required to synthesize the collagenous matrix and its supportive extrafibrillar proteoglycans. That is, metal cations increase the rate of chemical reactions without undergoing permanent changes themselves.

Collagenesis, or collagen biosynthesis, is a necessary process for the correction of damage to the skin caused by aging or other factors. Because the metal cations help promote collagen biosynthesis, the inclusion of metal cations in these embodiments of the transdermal delivery composition not only enhance penetration of the target drug through the stratum corneum and epidermis, but also provide a natural catalyst to the healing of the skin and to the biosynthesis and maturation of collagenous tissue.

The metal cations may be included in the transdermal delivery composition either in their free form or in a form in which they are combined with a polypeptide or protein. Non-limiting examples of suitable metal cations include iron (Fe), copper (Cu) and calcium (Ca). A polypeptide is any member of a class of compounds having low molecular weight and which yields two or more amino acids upon hydrolysis. Peptides form the constituent parts of proteins and will therefore breach the epidermal barrier and carry the metallic co-factors into the dermis. One exemplary transdermal delivery composition includes epidermal penetrants combined with metal cations in a pharmaceutically acceptable carrier to ensure bioavailability of the metal cations.

Metal cations play a role in cell proliferation in general, and in collagen biosynthesis in particular. Specifically, iron (Fe) is involved in the proliferation of cells such as skin fibroblast cells. Fe stimulates cell proliferation at the chromosomal and DNA replication step. Fe is also involved in cell proliferation through its role as a co-factor in cytochromal enzymes in mitochondria. Iron (Fe) is used as a catalyst in Fenton's reaction (i.e. the oxidation of certain acids using hydrogen peroxide and ferrous salts), which results in oxidative damage to cells, but also stimulates cell proliferation as a defense mechanism against destructive reactive oxygen species (ROS). Fe is also involved in several signal transmission enzyme systems, such as cAMP as well as proteases, which are required to remove old and/or damaged cellular components in anticipation of the generation of new cells using Fe once again.

Calcium (Ca) plays several roles in collagen biosynthesis. Collagen biosynthesis begins with the destruction and removal of existing collagen molecules, and can begin either in response to damage to the collagen molecules, or simply as a natural process of collagen biosynthesis. Destruction of existing collagen molecules can result from the actions of enzymes such as matrix metalloproteinases (MMPs). These enzymes have Ca co-factors. Furthermore, the destruction of existing collagen molecules is part of general cellular destruction, which can result from the actions of heat shock proteins. These enzymes also have Ca co-factors. Destruction of existing collagen is followed by the synthesis of new collagen molecules. Conversion of inactive collagen (procollagen) to active collagen (tropocollagen) is effected by the actions of two enzymes (N- and C-proteases). These two enzymes cleave the N- and C-terminals of procollagen to form tropocollagen. Both have Ca co-factors. Also, Ca is involved in general cell proliferation through its role in Ca channels, which are used to deliver stimulant factors (such as metal cations) to cells, and also through its role in the action of factors such as cAMP.

Copper (Cu) plays a more specific role in collagen biosynthesis. Its role is primarily related to the cross-linking of collagen molecules. Cu stimulates cross-linking of collagen molecules in two ways. First, Cu is a co-factor for the enzyme Lysyl oxidase, which catalyses the cross-linking process in collagen. Second, Cu is involved in the cross-linking of collagen through its action as a catalyst in free radical producing reactions (i.e. Fenton type reactions).

The metal cations may be present in the physiological system both in free form as well as in a bonded form, in which they are bonded to a polypeptide or a protein. Non-limiting examples of such proteins include: Calmodulin for Ca, Celeruplasmin for Cu, and Albumin or Desfroxamin for Fe.

In one embodiment, the transdermal delivery composition has a pH ranging from acidic to physiological, e.g. from about 3.0 to about 7.4. Permeation enhancers with pH values within this range are highly effective permeation enhancers and exhibit superior permeation abilities.

### EXPERIMENTAL EXAMPLE 1

### Penetration of the Transdermal Delivery Compositions

A study was performed to confirm that the inventive transdermal delivery compositions rapidly penetrate the human skin. In particular, the study was conducted to determine: 1) whether the transdermal delivery compositions penetrate the human skin; and 2) how long it takes for the transdermal delivery composition to penetrate the skin.

The “skin” used for the study was EpiDerm™ Skin Model (EPI-200×) (MatTek Corp.), a human skin equivalent. This skin equivalent includes normal, human-derived epidermal keratinocytes and normal, human-derived dermal fibroblasts which have been cultured to form a multilayered, highly differentiated model of the human dermis and epidermis. The tissues are cultured on specially prepared cell culture inserts using a serum free medium to attain levels of differentiation on the cutting edge of in vitro skin technology. The EpiDerm™ Skin Model closely parallels human skin, thus providing a useful in vitro means to assess percutaneous absorption or permeability.

A permeation device (EPI-100-PBS from MatTek Corp.) was used to measure percutaneous penetration of the preparations. The cell culture insert, which contained the EpiDerm™ tissue, was properly inserted into the permeation device.

Two groups of samples were tested. In the first group, a specimen cream, a control base, and a donor solution with no samples added as the negative control were tested. The specimen cream included a formulation of 2% by weight benzyl alcohol and 0.6% by weight lecithin organogel.

In the second group, a donor solution (phosphate buffer solution or “PBS”) with no samples added as a control, and donor solutions prepared containing four different concentrations (0.25 g/ml, 0.5 g/ml, 1 g/ml and 2 g/ml) of the specimen cream or the control base were tested. Neutral Red (0.001%) was added to give a red tinge to the donor solution.

Each sample was added to the permeation device containing the skin tissue, and the assembly was placed into the wells of a 6 well plate containing 3 ml of PBS. The assembly was moved to a fresh well containing 3 ml of PBS at the following intervals: 15 min, 30 min, 45 min, 60 min, 90 min, 120 min, 150 min, 180 min, 210 min, 240 min, 270 min, 300 min, 330 min, 360 min, 12 hrs, 24 hrs. After incubation, PBS from the 6 wells were collected in separate tubes, labeled and stored at −70° C. for further processing.

To determine if the specimen cream had penetrated through the epidermal layer, the samples (collected after incubation) were subjected to elemental analysis by analyzing selected time points and concentrations. The following 14 samples were analyzed:

Group 1 samples:

| | |
|---|---|
| Specimen cream sample | 15 min, 30 min, 60 min and 120 min |
| Control Base sample | 15 min, 30 min, 60 min and 120 min |
| PBS control | 15 min, 60 min, 120 min |

Group 2 samples:

| | |
|---|---|
| Donor solution sample (0.25 g/ml) | 15 min |
| Donor solution base (0.25 g/ml) | 15 min |
| PBS | 15 min |

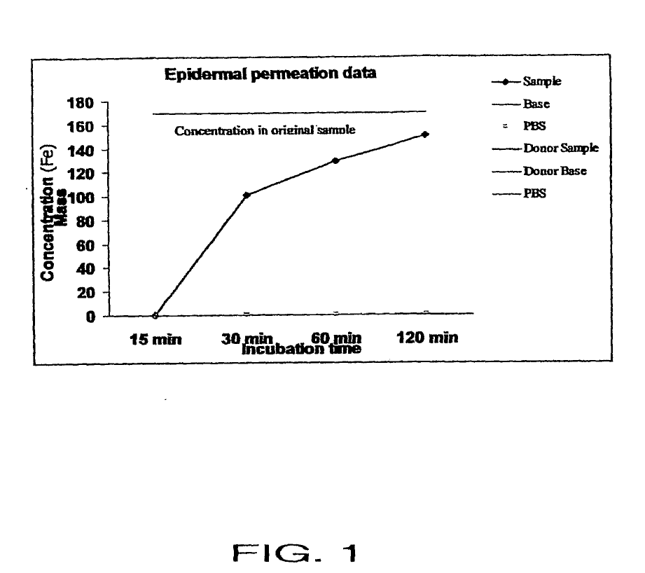

The samples were analyzed by a PIXE analyzer, which measured 74 elements in one run. Two elements were of primary interest, copper (Cu) and iron (Fe). The results of the PIXE analysis are shown in FIGS. 1 (Fe) and 2 (Cu). All skin tissue in this study were viable at the end of the study period after 120 hrs of incubation. The donor sample had a Fe concentration mass of 169.708 (straight line) and a Cu concentration mass of 3.132 (straight line).

The results of the PIXE analysis show that the specimen cream (i.e. the formulation of 2% by weight benzyl alcohol 2% and 0.6% by weight lecithin organogel) does penetrate the epidermis and it does so within 30 minutes of application. The specimen samples started showing an increase in the concentration mass of Fe starting at 30 min and reached a peak value in 120 min. Fe was undetectable in wells incubated with base or PBS. In addition, the specimen samples started showing an increase in the concentration mass of Cu starting at 30 min and reached a peak value in 120 min. Cu was undetectable in wells incubated with base or PBS. Thus, the compound is available to the deeper layers of the skin (especially dermal fibroblasts) within 30 minutes of its application to the epidermal surface.

### EXPERIMENTAL EXAMPLE 2

### Bioactivity of the Transdermally Delivered Agent

A second study was performed to determine whether the transdermally delivered compound remains bioactive. In particular, the second study was conducted to determine whether the delivered compound affects the dermal fibroblasts, and whether it induces procollagen synthesis in those cells.

Pro-collagen synthesis was measured by a real time PCR machine in human dermal fibroblasts (cell line purchased from Cambrex Bio Sciences Walkersville, Inc.) following exposure to the compound. In this study, a specimen cream including a formulation of 2% by weight benzyl alcohol and 0.6% by weight lecithin organogel was compared with a control base.

A real time PCR method was used to determine collagen message levels in the human dermal fibroblast cell lines exposed to the specimen cream at a concentration of 0.25 mg/ml and the control base at a concentration of 0.25 mg/ml. Cells incubated in media alone served as negative controls.

Absolute quantities of collagen were determined in the fibroblasts using real time RT-PCR analysis. Briefly, cDNA was prepared from the fibroblasts using a retroscript RT-PCR kit purchased from Ambion Inc. RT reactions without reverse transcriptase served as negative controls. Ten nanograms of cDNA was used as a template for the RT-PCR reaction. Forward primers, reverse primers and TaqMan® probes were purchased from Applied Biosystems (Foster City, Calif.). A collagen type 1 alpha 1 probe was labeled with the reporter dye, FAM (6-carboxyfluorescein) at the 5′ end and a non-fluorescent quencher dye at the 3′ end. The primers remained unlabeled. The master mix for the PCR reaction included 10 μl of universal master mix (from Applied Biosystems), 900 nM of each primer and 250 nM of each probe in a final volume of 20 μl. All PCR reactions were carried out in triplicate wells of a 96-well microamp optical plate (Applied Biosystems). Thermal cycling and data analyses were performed in an ABI Prism 7300 instrument (Applied Biosystems). A standard curve generated using different concentrations (10 ng, 1 ng, 0.1 ng, 0.01 ng and 0.001 ng) of collagen plasmid was used for quantitative determination of collagen mRNA in the samples.

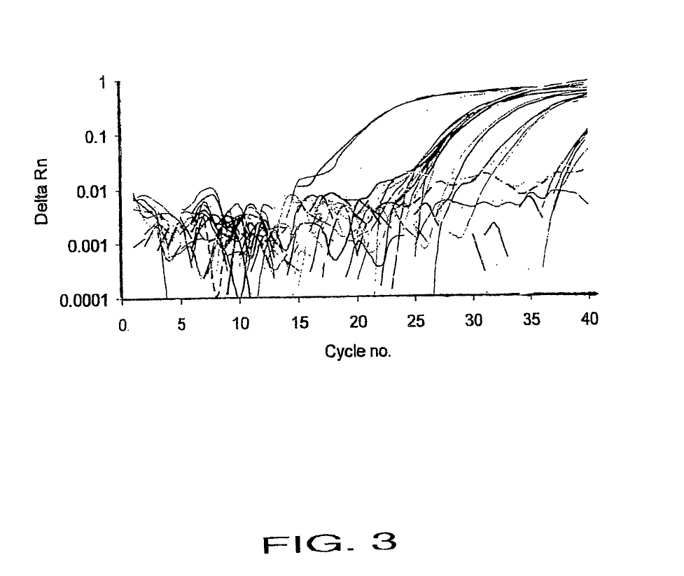

These analyses showed that exposure to the specimen cream induced the expression of collagen in human dermal fibroblasts within 30 minutes, as shown in FIG. 3. These results show that human dermal fibroblast cells began expressing pro-collagen within 30 min after exposure to the specimen sample. Control samples exposed to base alone did not express pro-collagen at this time point. Similar changes were not observed at 30 minutes when the base was applied to fibroblast cultures. Thus, these findings correlate with the penetration data and suggest that the specimen cream, after penetrating through the epidermal layer of the skin, can induce collagen synthesis in human dermal fibroblast cells.

In sum, the specimen cream (formulation of 2% by weight benzyl alcohol and 0.6% by weight lecithin organogel with Fe and Ca peptides), at a minimum concentration of 0.25 gm/ml, when applied to the epidermal surface of skin, penetrates through the epidermal layer and reaches the dermal layer within 30 minutes after application. In addition, human dermal fibroblasts produce collagen type 1 alpha 1 within 30 minutes after application of the specimen cream at a minimum concentration of 0.25 mg/ml.

Of material importance in fully evaluating this study is the fact that the 3-dimensional tissue constructs utilized are metabolically and mitotically active making these studies comparable to in-vivo studies.

II. Delivery of a Target Drug, Agent or Composition through the Epidermis

As noted above, according to some embodiments of the present invention, the inventive transdermal delivery compositions are applied to the skin prior to treatment with any target drug. In alternative embodiments, however, the target drug and the transdermal delivery composition may be combined in a single topical composition that is administered to the skin. In either case, the inventive transdermal delivery compositions may be used to deliver a wide variety of drugs and agents through the stratum corneum and epidermis to the dermis. Non-limiting examples of these drugs and agents include antioxidants, retinoids, botulina toxins (BOTOX®), anti-fungal medications and agents, anesthetics, anti-inflammatories, etc.

The target drug is generally selected based on the skin ailment to be treated. The inventive transdermal delivery compositions can be used to deliver a wide variety of target drugs to treat a wide variety of skin ailments. For example, the inventive transdermal delivery compositions can be used to deliver target drugs for the treatment of aged (either intrinsic or extrinsic) skin, xerosis of the skin, dry skin, wrinkles or other imperfections in the skin caused by aging or muscular contraction, irregular pigmentation or lightening of the skin, fungal infections of the skin, and pain in the skin caused by external factors such as insect bites or burns.

In addition to their many uses in delivering target drugs for the treatment of skin ailments, the inventive transdermal delivery compositions can be used simply as an alternative form of drug delivery, whether the drug is intended to treat a skin ailment or an ailment affecting another part of the body. One non-limiting example of such a use is the transdermal delivery of anti-inflammatories. Transdermal delivery of anti-inflammatories has many benefits, which are described in more detail below. However, one great advantage of transdermal delivery of these drugs is the avoidance of the adverse side effects normally associated with oral administration.

As noted above, in some embodiments, the target drug, agent or composition is combined with a transdermal delivery agent or composition to form a single topical composition. In addition to the target drug, agent or composition and the transdermal delivery agent or composition, these topical compositions include a pharmaceutically acceptable carrier. In one embodiment, for example, a topical composition includes a therapeutically effective amount of the target drug to be administered, permeation enhancer(s) active at a pH ranging from about 3.0 to about 7.4 to enhance flux of the compound, and a topical pharmaceutically acceptable carrier suitable for topical or transdermal administration. Such a composition may have a pH ranging from about 3.0 to about 7.4.

In one embodiment, the topical pharmaceutically acceptable carrier includes: dimethyl sulfoxide; lecithin; ethanol; an isopropyl ester of a long-chain fatty acid selected from isopropyl palmitate, isopropyl stearate and isopropyl myristate; and a nonionic surfactant with free hydroxyl groups.

In one exemplary embodiment, the isopropyl ester of a long-chain fatty acid is isopropyl palmitate. The nonionic surfactant may be an ethylene oxide/propylene oxide block copolymer. One non-limiting example of a suitable ethylene oxide/propylene oxide block copolymer is Pluronic F127 (commercially available from BASF (Mount Olive, N.J.)). Other suitable nonionic surfactants are known in the art and may also be used. Non-limiting examples of such surfactants include ethoxylated ethers and ethoxylated esters having a carbon chain length ranging from 8 to 22 carbon atoms.

The topical pharmaceutically acceptable carrier may further include: water; propylene glycol; carbopol; an octyl ester of a long-chain fatty acid selected from octyl palmitate, octyl stearate, and octyl myristate; silicone fluid; cetearyl alcohol; a suitable buffer capable of buffering the pH of the composition to a value ranging from about 3.0 to about 7.4; and at least one non-sensitizing preservative.

One non-limiting example of a suitable silicone fluid is a silicone fluid with a viscosity of about 200 cps.

One non-limiting example of a suitable preparation of carbopol is Carbopol 940. Other carboxypolymethylene polymers, such as Carbomer polymers, may also be used. One non-limiting example of a suitable buffer is triethanolamine. However, any buffer capable of buffering the topical pharmaceutically acceptable carrier to a pH ranging from about 3.0 to about 7.4 may be used.

The octyl ester of a long-chain fatty acid may be selected from octyl palmitate, octyl stearate, and octyl myristate. In one embodiment, the octyl ester is octyl palmitate.

The topical pharmaceutically acceptable carrier may optionally further include other ingredients. For example, the topical pharmaceutically acceptable carrier may further comprise an acid (as needed) in a quantity sufficient to adjust the pH of the composition to a value ranging from about 3.0 to 7.4. In one exemplary embodiment, the acid is an organic acid with a carbon chain ranging from 2 to 22 carbons in length. In another embodiment, the acid is a monocarboxylic, dicarboxylic or tricarboxylic acid. For example, in one embodiment, the acid is citric acid.

In addition, the topical pharmaceutically acceptable carrier may further include a surface-coated starch polymer. One non-limiting examples of a suitable surface-coated starch polymer is Dryflo PC (commercially available from National Starch).

The topical pharmaceutically acceptable carrier may also further include a long-chain fatty acid isopropyl ester selected from isopropyl palmitate, isopropyl myristate, and isopropyl stearate. In one embodiment, the long-chain fatty acid isopropyl ester is isopropyl palmitate.

According to another embodiment, the topical pharmaceutically acceptable carrier may further include a mixture of glyceryl stearate and PEG-100 stearate. One non-limiting example of a suitable mixture is Arlacel 165.

In yet another embodiment, the topical pharmaceutically acceptable carrier further includes a long-chain fatty acid selected from palmitic acid, stearic acid, and myristic acid. In one embodiment, for example, the long-chain fatty acid is stearic acid.

According to still another embodiment, the topical pharmaceutically acceptable carrier further includes a caprylic/capric triglyceride. One non-limiting example of a suitable caprylic/capric triglyceride is Miglyol 812.

The topical pharmaceutically acceptable carrier may also further include cetearyl alcohol.

In another embodiment, the topical pharmaceutically acceptable carrier may further include a caprylic/capric stearyl triglyceride. One non-limiting example of a suitable caprylic/capric stearyl triglyceride is Softisan 378.

According to yet another embodiment, the topical pharmaceutically acceptable carrier may further include a fragrance. Non-limiting examples of suitable fragrances include natural lavender and chamomile oils. Other fragrances are well known in the art and can also be used.

The non-sensitizing preservative in the topical pharmaceutically acceptable carrier may include methylparaben, ethylparaben, propylparaben, butylparaben, diazolidinyl urea and mixtures thereof. In one embodiment, for example, the non-sensitizing preservative includes methylparaben, propylparaben, and diazolidinyl urea. One non-limiting example of a suitable preparation of diazolidinyl urea is Germall 2.

The topical pharmaceutically acceptable carrier may include a variety of other ingredients well known in the art. For example, other lipid-soluble components can be used in addition to, or in place of, the caprylic/capric triglycerides. Non-limiting examples of such components include steareth-2, steareth-21, polyglyceryl-3 beeswax, branched-chain carboxylic acid esters of branched-chain alcohols, acrylates/C₁₀-C₃₀ alkyl acrylates cross-polymers, methylgluceth-20, glyceryl esters of long-chain fatty acids, hydrogenated vegetable oil, squalane, C₁₂-C₁₅ alkylbenzoate, di-C₁₂-C₁₅ alkylfumarate, cholesterol, lanolin alcohol, octyldodecanol, isostearic acid, branched-chain neopentanoates, arachidyl esters of short-chain carboxylic acids, jojoba oil, myristyl esters of long-chain fatty acids, bisabolol, hydrogenated jojoba oil, jojoba esters, methylgluceth-20 sesquistearate, PPG-14 butyl ether, PPG-15 stearyl ether, PPG-1-isoceteth-3-acetate, laureth-2-benzoate, diisostearyl dimer dilinoleate, long-chain cis-monounsaturated fatty acid esters of medium-chain alcohols, medium-chain saturated carboxylic acid esters of long-chain alcohols, hydrogenated soy glycerides, long-chain fatty acid esters of cetyl alcohol, palm kernel oil, and palm oil. Non-limiting examples of suitable branched-chain carboxylic acid esters of branched-chain alcohols include isononyl isononanoate, isodecyl isononanoate, isooctyl isononanoate, isononyl isooctanoate, isodecyl isooctanoate, isooctyl isooctanoate, isononyl isodecanoate, isooctyl isodecanoate, and isodecyl isodecanoate. Non-limiting examples of suitable glyceryl esters of long-chain fatty acids include glyceryl monostearate, glyceryl monopalmitate, and glyceryl monoarachidate. Non-limiting examples of suitable branched-chain neopentanoates include octyldodecyl neopentanoate, heptyldodecyl neopentanoate, nonyldodecyl neopentanoate, octylundecyl neopentanoate, heptylundecyl neopentanoate, nonylundecyl neopentanoate, octyltridecyl neopentanoate, heptyltridecyl neopentanoate, and nonyltridecyl neopentanoate. Non-limiting examples of suitable arachidyl esters of short-chain carboxylic acids include arachidyl propionate, arachidyl acetate, arachidyl butyrate, and arachidyl isobutyrate. Non-limiting examples of suitable myristyl esters of long-chain fatty acids include myristyl myristate, myristyl laurate, and myristyl palmitate. Non-limiting examples of long-chain fatty acid esters of cetyl alcohol include cetyl palmitate, cetyl stearate, and cetyl myristate.

In addition, the topical pharmaceutically acceptable carrier may further include ingredients generally used in cosmetics and skin preparations. Non-limiting examples of such ingredients include plant extracts, such as horsetail extract, horse chestnut extract, rose extract and lavender extract. Other non-limiting examples of suitable ingredients include long-chain fatty acid esters of retinol or retinol derivatives or analogues, such as those in which the acyl moiety of the ester is selected from myristic acid, palmitic acid, and stearic acid. Yet other non-limiting examples of suitable ingredients include sunscreens, such as those selected from octyl methoxycinnamate, p-aminobenzoic acid, ethyl p-aminobenzoate, isobutyl p-aminobenzoate, glyceryl aminobenzoate, p-dimethylaminobenzoic acid, methyl anthranilate, menthyl anthranilate, phenyl anthranilate, benzyl anthranilate, phenylethyl anthranilate, linalyl anthranilate, terpinyl anthranilate, cyclohexenyl anthranilate, amyl salicylate, phenyl salicylate, benzyl salicylate, menthyl salicylate, glyceryl salicylate, dipropyleneglycol salicylate, methyl cinnamate, benzyl cinnamate, .alpha.-phenyl cinnamonitrile, butyl cinnamoylpyruvate, umbelliferone, methylacetoumbelliferone, esculetin, methylesculetin, daphnetin esculin, daphnin, diphenylbutadiene, stilbene, dibenzalacetone, benzalacetophenone, sodium 2-naphthol-3,6-disulfonate, sodium 2-naphthol-6,8-disulfonate, dihydroxynaphthoic acid, salts of dihydroxynaphthoic acid, o-hydroxybiphenyldisulfonates, p-hydroxybiphenyldisulfonates, 7-hydroxycoumarin, 7-methylcoumarin, 3-phenylcoumarin, 2-acetyl-3-bromoindazole, phenylbenzoxazole, methylnaphthoxazole, arylbenzothiazoles, quinine bisulfate, quinine sulfate, quinine chloride, quinine oleate, quinine tannate, 8-hydroxyquinoline salts, 2-phenylquinoline, hydroxy-substituted benzophenones, methoxy-substituted benzophenones, uric acid, vilouric acid tannic acid, tannic acid hexaethylether, hydroquinone, oxybenzone, sulisobenzone, dioxybenzone, benzoresorcinol, 2,2′,4,4′-tetrahydroxybenzo-phenone, 2,2′-dihydroxy-4,4′-dimethoxybenzophenone, octabenzone, 4-isopropyldibenzoylmethane, butylmethoxydibenzoylmethane, etocrylene, and 4-isopropyldibenzoylmethane.

One non-limiting and exemplary formulation of the topical pharmaceutically acceptable carrier is shown in Table 1 below.

TABLE 1
Exemplary formulation of a topical
pharmaceutically acceptable carrier

| INGREDIENT | % BY WEIGHT |
|---|---|
| Propylene glycol | 2.23 |
| Carbopol | 1.12 |
| Surface coated starch polymer | 0.56 |
| Octyl palmitate | 1.12 |
| Isopropyl palmitate | 2.23 |
| Silicone fluid | 2.23 |
| Glyeryl stearate/PEG-100 stearate | 2.23 |
| Cetearyl alcohol | 1.12 |
| Stearic acid | 0.56 |
| Triethanolamine | 0.28 |
| Caprylic/capric triglyceride | 2.23 |
| Caprylic/capric stearyl triglyceride | 0.56 |
| Natural lavender/chamomile oils | 0.22 |
| Methylparaben | 0.22 |
| Propylparaben | 0.06 |
| Diazolidinyl urea | 0.22 |
| Water | q.s. to 100 |

The compositions, devices and methods of the present invention provide enhanced transdermal delivery, increasing efficiency and decreasing pain and discomfort normally associated with more invasive treatments, such as drug injections. Other possible complications with injections include localized swelling or edema, capillary hemorrhage and inflammation.

Current approaches to transdermal drug delivery are not particularly effective. They are typically device or patch-dependent, rely on in-vitro models alone (i.e. they have limited relevance) and are limited in dose (less than 10 mg per day), polarity (primarily lipophilic) and drug class (peptides are excluded). Given these limitations, only a few drugs have been successful using these approaches, e.g. nitroglycerin, scopolamine, clonidine, estrogen, testosterone and nicotinic acid.

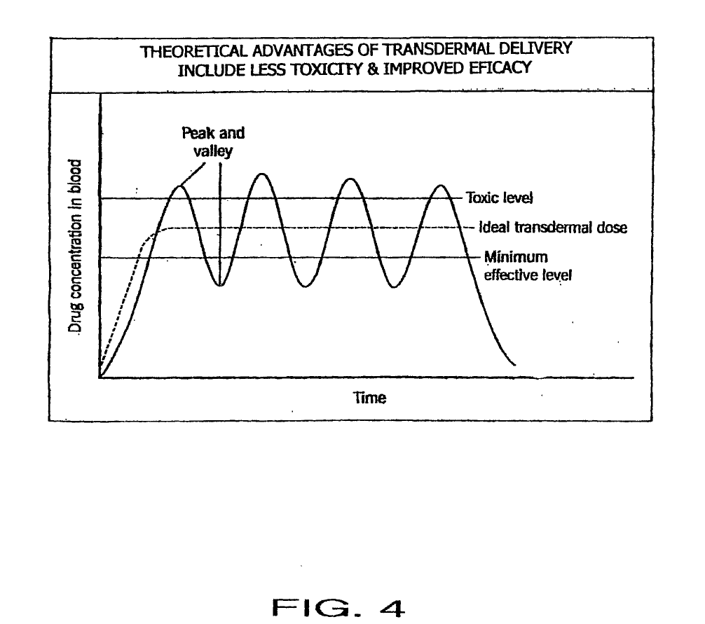

However, these complications are not associated with the compositions, devices and methods of the present invention. Rather, the transdermal delivery compositions and methods according to the present invention improve patient compliance, have improved efficacy (i.e. continuous release), have reduced toxicity (i.e. no peaks and a lower total absorbed dose) and decreased dosing frequency. These effects are achieved due to reductions in the “peaks” and “valleys” associated with bolus therapy (See FIG. 4). In addition, the compositions and methods of the present invention bypass hepatic first-pass metabolism, avoid local gastrointestinal side effects, avoid painful injections and decrease costs to the patient due to decreases in the total dose and dosing frequency.

Also, the speed of transport of the inventive compositions is comparable or even more rapid than that of other means of delivery, such as injection. Although speed of transport is important, the compositions according to the present invention also provide for the comprehensive transport of the target agent or composition, thereby ensuring that the agents are bioavailable. Rapid transport combined with bioavailability enables the inventive compositions to provide efficacious bioactivity of the agents with dosimetry at a safer level than other delivery methods. The inventive compositions and methods also target specific biochemical mechanisms and take advantage of the localization and relative importance of the steps leading to the generation and maintenance of functional stratum corneum extracellular lamellae.

Compositions according to certain embodiments of the present invention may be in the form of creams, ointments or saturated absorbent cloths. The compositions may be placed over the target site, thereby avoiding inadvertent diffusion of the composition into an unwanted site.

### III. Compositions for Promoting Collagen Synthesis for the Treatment of Aging, Xerosis or Dry Skin

One skin malady that affects all people is aging. Aging can include chronological or intrinsic aging, or photo-aging (solar or extrinsic aging). Photo-aging is caused by exposure of the skin to the sun. Such exposure causes certain changes to occur in the exposed skin. These changes are generally referred to as solar aging and can include damage to both the outer, superficial layers of the skin as well as to the deeper, structural and supportive layers of the skin.

Aging, whether intrinsic or extrinsic, results in the up-regulation or increase in three specific, destructive enzymes found in the skin. These enzymes are collectively referred to as matrix metalloproteinases (“MMP”) and include collagenase, 92 kD gelatinase and stromelysin-1. The actions of these enzymes combine to cause the degradation and cleavage of collagen and elastin molecules and the destruction of other supportive elements of the skin. Intrinsic aging also triggers action by MMPs in the skin.

Significantly, it takes only a single exposure to the ultraviolet radiation of the sun the induce such action by these MMPs in the skin. Exposure to levels of UV light that cause no detectable sunburn induce the expression of matrix metalloproteinases (MMPs) in keratinocytes (KC) in the outer layers of the skin, as well as fibroblasts (FB) in connective tissue. These MMPs degrade collagen in the extracellular matrix of the dermis. The extent of matrix destruction is limited by the simultaneous induction of the tissue inhibitors of MMPs-1 (TIMP-1), which partially inhibit the activity of MMPs. The breakdown of collagen is followed by synthesis and repair, which as with all types of wound healing, is imperfect and chaotic and leaves subtle clinically undetectable deficits in the organization or composition of the extracellular matrix, or both. Matrix damage, followed by imperfect repair, occurs with each ensuing exposure to the sun, leading to the accumulation of an altered matrix (solar scar) and eventually, observable photo-aging, such as wrinkles

Damage caused by xerosis and aging (intrinsic and extrinsic) of the skin results from diminished dermal collagen and elastin fibers and supportive non-fibrillar ground elements, i.e. proteoglycans (“PGs”). Dermal collagen volume linearly declines throughout a person's life, and declines to a greater degree in photo-aged skin than in intrinsically aged skin.

Besides collagen, elastin is the other major protein element in the skin, and provides elasticity to the skin. Cross-linking this protein allows the elastic fibers to stretch by 100% or more and still return to their original form. This elastic function of elastin complements the function of collagen, which is to impart tensile strength to the skin. Aging, and particularly photo-aging, also causes degradations in elastin and its cross-links, resulting in a loss of skin elasticity.

Comprehensive losses of collagen and elastin along with the accompanying proteoglycans (PGs) leads to damage to associated blood supply and xerosis. These effects combine to form the true foundation for commonly observed changes in the skin due to aging. These physical changes result from the skin's lack of structural integrity and a relative instability in the dermal-epidermal junction, which is constantly affected by the natural forces of gravity. The combination of these changes cause textural changes, sallow appearance, fine lines, wrinkles and furrows normally associated with aging.

In xerosis, which may be induced by intrinsic or extrinsic aging, the structural integrity of the skin, which is a manifestation of the support provided by the underlying dermis, is altered. The collagenous matrix in the dermis is damaged, and the extrafibrillar matrix and epidermal barrier to water loss is altered. Repair or reversal of this damage requires collagen biosynthesis. Trans-retinoic acids (e.g. tretinoin) can repair photo-aged skin.

However, collagen biosynthesis alone will not remedy this condition. Maturation of the newly generated collagen and epidermal barrier repair must take place before the skin can be restored to its pre-trauma condition.

Additionally, the role of the ground substance of the skin has not historically been properly appreciated. Although the ground substance has been presumed biologically structured and inert, it is in fact molecularly and structurally diverse, highly organized, and biologically active. The role of the ground substance is frequently overlooked in existing treatment options for solar-damaged or aged skin.

To better treat xerosis and aging of the skin, certain embodiments of the present invention address both collagen biosynthesis and collagen maturation. Some embodiments of the present invention also address the role of the ground substance and its interaction with collagen.

Collagen maturation can be defined as the process by which the fragile, soluble fibrils of collagen change into strong, insoluble fibers as they proceed from a disorganized, random, and not very useful arrangement to an organized, oriented structure providing mechanical strength to tissue, e.g. skin. The critical feature of this maturation is cross-linking.

Changes in the solubility of collagen fibers occur in newly formed collagen as it is deposited to form connective tissue structures in the body. Simultaneously, the tensile strength of fibers increases dramatically and continues to increase even after the fibers have become insoluble in neutral salt solutions. The physical properties of newly synthesized collagen fibers are affected by cross-linking.

Mammals have at least 33 genetically distinct polypeptide chains comprising at least 20 distinct collagen types that occur in different tissue in the same individual. Of these, the one generally occurring in skin is referred to as Type I collagen. Type I collagen has the chain composition [α1(1)₂α2(1)]. A single molecule of Type I collagen includes three polypeptide chains with an aggregate molecular mass of about 285 kD. It has a rod-like shape with a length of about 3000 Å and a width of about 14 Å.

Collagen has a distinctive amino acid composition. Nearly one-third of its residues are glycine and another 15-30% of them are proline and 4-hydroxyproline residues. Other modified residues, namely 3-hydroxyproline and 5-hydroxylysine residues, also occur in collagen but in smaller amounts. These nonstandard hydroxylated amino acids are not incorporated into collagen during polypeptide synthesis, but are produced by post-translational modification. Proline residues are converted to hydroxyproline in reactions catalyzed by the enzyme prolyl hydroxylase. The 4-hydroxyproline residues impart stability to collagen, likely through intramolecular hydrogen bonds that involve bridging water molecules. Prolyl hydroxylase requires ascorbic acid (vitamin C) for activity.

The amino acid sequence of bovine collagen α1(1), which is similar to that of other collagens, consists of monotonously repeating triplets of the sequence Gly-X-Y over a continuous 1011-amino acid stretch of this 1042-residue polypeptide chain. In this repeating sequence, X is often proline and Y is often 4-hydroxyproline. The restriction of 4-hydroxyproline to the Y position in this repeating pattern stems from the specificity of prolyl hydroxylase. The modified amino acid 5-hydroxylysine is also similarly restricted to the Y position in this repeating pattern. X-ray diffraction has confirmed that collagen has a triple helical structure. The three polypeptide chains are parallel and wind around each other with a gentle, right-handed rope-like twist to form this triple helical structure. An individual collagen polypeptide helix has 3.3 residues per turn and a pitch of 10.0 Å. The three polypeptide chains are staggered so that the Gly, X, and Y residues in the repeating three-amino-acid sequence occur at similar levels. The staggered peptide groups are oriented so that the N—H group of each glycine residue makes a strong hydrogen bond with the carbonyl oxygen of each residue in the X position in a neighboring chain. The bulky and relatively inflexible Pro and 4-hydroxyproline residues confer rigidity on the entire assembly. This triple helical structure is responsible for its characteristic tensile strength. As with the twisted fibers of a rope, the extended and twisted polypeptide chains of collagen convert a longitudinal tensional force to a more easily supported lateral compressional force on the almost incompressible triple helix. This occurs because the oppositely twisted directions of collagen's polypeptide chains and triple helix prevent the twists from being pulled out under tension, as in ropes and cables.

Collagen is further organized into fibrils. These fibrils typically have a periodicity of 680 Å and a diameter of 100 to 200 Å. X-ray fiber diffraction has shown that the molecules in fibrils of Type I collagen are packed in a hexagonal array. The collagen molecules in the array are staggered parallel to the fibril axis. The driving force, energetically, for the assembly of collagen molecules into a fibril is apparently provided by the added hydrophobic interactions within the fibrils.

Collagen also contains covalently attached carbohydrates in amounts that range from about 0.4% to about 12% by weight, depending on the collagen's tissue of origin. The carbohydrates consist mostly of glucose, galactose, and their disaccharides. They are covalently attached to collagen at its 5-hydroxylysine residues by specific enzymes. The function of the carbohydrates is not completely known, but they may be involved in directing fibril assembly.

Additional structural stability is provided in collagen by covalent cross-linking between the collagen fibrils. The cross-linking is derived from lysine and histidine side chains in reactions catalyzed by the enzyme lysyl oxidase. Lysyl oxidase is a Cu(II)-containing metalloenzyme. In the absence of copper, the formation of lysyl and hydroxylysyl aldehydes is blocked, thereby preventing the cross-linking of collagen as well as of elastin. The first step is the oxidation of lysine residues to allysine. The next step is the aldol condensation of two allysine residues to form allysine aldol. The third step is the reaction of the allysine aldol with histidine to form an aldol-histidine product. This, in turn, can react with 5-hydroxylysine to form a Schiff base (an imine bond), which cross-links four side chains. The cross-linked product is histidinodehydrohydroxymerodesmosine. This hierarchical structure is important in understanding the process of collagen maturation.

Single molecules of collagen are referred to as tropocollagen. When tropocollagen first aggregates, the force that holds the chains of tropocollagen together in their inherent arrangement is due to electrostatic bonds. When tropocollagen is first formed from procollagen, the individual α chains are held together only by hydrogen bonds. However, as compared with electrostatic bonds, hydrogen bonds are relatively weak.

Further, during maturation, covalent bonds are formed between the α1 and α2 chains. This is termed an intramolecular bond because it occurs within a single tropocollagen molecule. The formation of an intramolecular bond does not alter the solubility of collagen, but it does make the molecule much more stable and possibly increases its resistance to attack by enzymes. One of the most stable cross-links is the intermolecular cross-link resulting from a shift of the double bonds in the Schiff base to form a ketone. This is the major force holding fibrils and their bundles together. Their presence is the chief contributing factor in the tensile strength of collagen. The formation of intramolecular and intermolecular cross-links involving aldehyde groups occurs early in the formation of collagen fibrils. Seven distinct cross-links have been reported in collagen, all of them dependent on oxidative de-amination of lysine and hydroxylysine residues.

There are other types of bonding that further stabilize the collagen matrix. One important type of bonding that is frequently overlooked is electrostatic bonding among the protein-polysaccharides of the amorphous ground substance. This plays a role in the physical properties of the collagen fibril and may regulate the size of the fibril. The fibrils become covalently linked to glycoproteins. It has been suggested that fibers are formed outside of the cell in a matrix that includes a variety of mucopolysaccharides, glycoproteins, and protein polysaccharides. Most of the sulfated mucopolysaccharides are present in the tissue in combination with protein. The high molecular weight hyaluronic acid, which is free, facilitates the proteoglycans to imbibe water. This permits the matrix to swell and support the collagen fibers.

This dermal fiber network and cells are embedded in an amorphous extrafibrillar material that binds water and provides the hydrated consistency of the skin. While previously presumed to be biologically unstructured and largely metabolically inert, this “ground substance” is actually molecularly and structurally diverse, highly organized and biologically active. The extrafibrillar matrix contains a number of proteoglycans and glycoproteins, hyaluronic acid, and water. Its functions vary and are adapted to the biological needs of each tissue type. For example, during embryonic development, water binding proteoglycans and glycosaminoglycans (“GAGs”) form a hydrated milieu for cell migration and proliferation. During development and tissue remodeling, glycoproteins of the extrafibrillar matrix are essential for formation of the correct tissue architecture and function as a biologic humectant.

GAGs are polysaccharides of sulfated and acetylated sugars with negative charges that bind large quantities of ions and water. Usually GAGs are bound to proteins with a serine hydroxyl group and form proteoglycans. However, the most prominent and ubiquitous protein-free GAG is hyaluronic acid, a giant polysaccharide composed of thousands of N-acetylglucosamine/glucuronic acid disaccharides.

Proteoglycans differ remarkably in their protein content and the number, type, and length of their GAG side-chains. Four different proteoglycan-bound GAGs are known: chondroitin sulfate, dermatan sulfate, keratan sulfate, and heparan sulfate. The structures of these proteoglycan-bound GAGs are known. Chondroitin sulfate occurs in two forms: chondroitin 4-sulfate and chondroitin 6-sulfate. Dermatin sulfate, which occurs frequently in skin, differs from chondroitin 4-sulfate only by the inversion of the configuration about C5 of the β-D-glucuronic residues to form α-L-iduronnate. Keratin sulfate contains alternating β-(1→4) linked D-galactose and N-acetyl-D-glucosasmine-6-sulfate residues. Heparan sulfate resembles heparin in its composition, and consists predominantly of alternating α(1→4) linked residues of D-iduronate-2-sulfate and N-sulfo-D-glucosamine-6-sulfate, like heparin, but has fewer N- and O-sulfate groups and more N-acetyl groups. Versican is the most important proteoglycan in the dermis, as it aggregates with hyaluronic acid and binds with large amounts of water. It is synthesized by fibroblasts in collagen bio-synthesis, is associated with the elastic fiber system and forms huge complexes with hyaluronic acid, which provides skin with its tautness.

Proteoglycans (“PGs”) are ubiquitous, non-fibrillar molecules. They form a heterogeneous group of protein-carbohydrate complexes, which serve several functions. For example, PGs function as adhesive molecules, providing an inflammatory cell role and ensuring the tensile strength of collagen fibers by means of another form of structural cross-linking, i.e. electrostatic bonding. PGs also play a primary role in the attraction and binding of great amounts of water responsible for tissue hydration. This water percolates anteriorly through the skin, as needed, to provide the supple, moist, plump skin prevalent in youth. A relative decrease in PGs is, in some measure, the basis for dry skin or xerosis associated with aging.

During disease or trauma, such as solar injury, GAG turnover is greatly enhanced. It is at this time that it becomes critical to replace these matrix components, particularly the GAGs, which seem to be most vulnerable to degradation. The tissue specific GAGs require a source of inorganic sulfur for their synthesis. Non-limiting, suitable sources of sulfur, according to the present invention, include the sulfur-containing amino acids (SAAs), e.g. cysteine and methionine. These are suitable sources of sulfate for the de novo synthesis of GAGs. These compounds are rapidly converted into free sulfates before or after absorption.

Of previously unrecognized significance in the synthesis of GAGs is that the recommended dietary allowance (RDA) for SAAs (e.g. methionine and cysteine), may in fact underestimate the bodily needs for these mutually complementary essential nutrients, particularly during periods of increased synthesis of GAGs. Such periods of increased synthesis of GAGs are likely to occur in individuals who have suffered solar or other skin damage, are aged, or are subject to other conditions affecting the integrity of the skin. Therefore, in addition to being building blocks for proteins such as collagen, the SAAs are the primary source of sulfur used in the synthesis of many key metabolic intermediates as well as GAGs (the main components of the extracellular matrix). According to one embodiment of the present invention, SAAs are included in the treatment of solar-damaged tissue, as they facilitate dermal hydration, which aids in filling and plumping of the overlying tissue, substantially reducing the appearance of rhytides.

As described above, cross-linking after fibril formation is extremely important as it is responsible for the mechanical properties of collagen, particularly tensile strength. Given that covalent cross-linking is so important in collagen maturation and in rebuilding tensile strength during wound recovery, some embodiments of the present invention promotes the formation of cross-links during treatment of wounds by introducing cross-linking agents, thereby speeding up the increase in tensile strength. This introduction of cross-linking agents increases the rate of increase in tensile strength not only in incisional wounds, but also in wounds caused by solar damage.

Because copper is directly involved in the cross-linking process, some embodiments of the present invention include it in a topical composition for the treatment of xerosis and aging of the skin. The addition of copper to the topical composition yields enhanced skin firmness of the skin. However, making the copper biologically available to the collagen biosynthetic process in the dermis poses a challenge. The mere presence of topically administered copper does not result in a significant influx of copper into the dermis without enhancing its permeability through the epidermis such as through the use of a transepidermal delivery agent or penetrant.

Innovations in laser, light and radiofrequency devices have improved therapeutic efficacy and safety in the treatment of solar aging with an ever-increasing number of medical and aesthetic indications. Some notable improvements include: (1) an expansion in the use of specific photonic wave lengths, pulse durations and cooling strategies; (2) the introduction of non-ablative rejuvenation techniques, including radiofrequency, intense pulsed light and other light-based modalities, such as photodynamic therapy and light emitting diode devices; (3) the use of combinations of laser, light, and radiofrequency technologies, which have provided dermatologic laser surgeons with expanded capabilities.

Despite such developments, safety and efficacy remain the primary concerns. Some complications remain commonly encountered with these modalities, in spite of the improvements in optical technology and the refinement of existing devices. The paradoxical issue is the relative ineffectiveness of these non-ablative techniques when the risks and complications are avoided. For example, while the use of so-called non-ablative processes has been driven by the desire to reduce down-time and associated risks and complications, clinical improvements in photo-aged skin has also diminished. This is evidenced by the fact that it now takes repeated, phasic treatment processes to obtain anywhere near the dramatic skin resurfacing results observed from older, more invasive treatment modalities.

These extraneous attempts to reverse changes caused by aging cause traumas leading to a wound repair cascade. This repair response is superimposed on the trauma caused by the sun and culminates in collagen synthesis and fibrosis. This duplicate attempt at collagen biosynthesis is frequently chaotic in nature and delays clinical improvement.

Collaterally associated with extrinsic and intrinsic aging is dry skin or xerosis. Dry skin or xerosis is ubiquitous, present in most individuals over the age of 60, and is one critical sign of solar damage. There are many proposed treatments for such a condition.

Dry skin is caused, at least in part, by a defective superficial epidermal barrier function allowing hydrated skin to lose its water through the epidermis into the environment. This is known as transepidermal water loss (“TEWL”). Dry skin (xerosis, exsiccosis, asteatosis) refers to a dry, rough, and scaly quality of the skin, which may result from both exogenous and endogenous causes: for example, dry climate, excessive exposure to water, alkali and detergents, marasmus and malnutrition, renal insufficiency, hemodialysis and hereditary conditions, such as ichthiosis vulgaris and atopy. However, the most common cause is aging, whether intrinsic or extrinsic. Dry skin appears most commonly in the sixth decade of life. Extrinsic or solar aging is another common cause of dry skin and results from the exposure of the skin to the UV rays of the sun. Ambient and lifestyle factors also play an important role in the appearance of xerosis.

The association of xerosis with aging appears to be directly related to the up-regulation of enzymes (such as specific lipases) which break down intercellular lipids, resulting in degradation of the critical epidermal barrier to water loss.

Dry skin occurs in nearly everyone over the age of 60, but to a variable degree, and is often unnoticed. Its severity is strongly linked to exogenous factors: for example, it is found more often in dry climates, during the winter months, and in persons who shower or bathe too often. However, dry skin is also found in individuals who are unable or unwilling to carry out proper skin care. Asteatosis occurs world-wide and may affect men more often than women.

Asteatotic eczema results from the dispositional irritability of dry skin plus exogenous triggers, including contact sensitivity to ingredients of topical preparations. Asteatosis is a cause of “nummular eczema.”

Xerosis of aging skin is not caused by deficient sebum production (e.g. children's skin is smooth even though sebum production is physiologically low). Rather, xerosis is caused by a complex dysfunction of the epidermal barrier layer.

There are three intercellular lipids involved in the epidermal barrier function: sphingolipids, free sterols and free fatty acids. In addition, it is believed that the lamellar bodies (i.e. Oldland bodies, membrane coating granules, cementsomes) containing sphingolipids, free sterols and phospholipids play a key role in the barrier function and are essential for trapping and preventing excessive water loss. These lipids are necessary to the epidermal barrier function since solvent extraction of these chemicals leads to xerosis to a degree directly proportional to the amount of lipid removed. The major lipid (by weight) found in the stratum corneum is ceramide, which becomes a sphingolipid when glycosylated via the primary alcohol of sphingosine. Ceramides possess the majority of the essential long-chain fatty acids (EFAs) of the skin, such as linoleic acid.

Skin suffering from xerosis has decreased intercellular lipids, is deficient in all key stratum corneum lipids, has an altered ratio of fatty acids esterified to ceramide 1 (of the 7 Cer classes, acylceramides, Cer 1 is the epidermal lipid known to be important for the epidermal barrier), has persistent corneodesmosomes, and prematurely expresses involucrin and the cornified envelope, resulting in corneocyte retention and marked impairment of barrier recovery subsequent to perturbation. Most importantly, the water-binding capacity of the horny layer is reduced owing to decreased synthesis of “natural moisturizing factor” (NMF), a hygroscopic mixture of amino acids (degradation products of profillagrin), urea and other compounds. Consequently, the horny layer dries out, loses its pliability and forms small cracks, which render the skin surface dull, rough and scaly.

Aggravating ambient factors include low relative humidity, low temperatures and chronic UV damage. In addition, damage to the horny layer may result from excessive use of soaps or surfactants (bath foams), habitual scrubbing, and washing out of (the water soluble) NMF by prolonged exposure to water (e.g. by frequent showering).

If mild, xerosis is asymptomatic, but if more pronounced, the skin conveys unpleasant sensations, such as itching and stinging. These sensations may be directly caused by stimulation of cutaneous nerve fibers. Inflammation is enhanced by the release of pro-inflammatory cytokines secondary to barrier perturbation, mechanical factors (scratching, rubbing) and the application of irritating and sensitizing substances contained in topical preparations, perfumed soaps, shower gels, etc.

Xerosis generally first arises on the shins and may remain limited to this area. Later, the xerosis may spread to the thighs, proximal extremities and trunk, but typically spares the face and neck as well as the palms and soles. Xerosis develops insidiously over many years, whereas asteatotic eczema often has a more sub-acute to acute onset.

Xerotic skin is dry and dull, with fine bran-like scales, which may be released as powdery clouds when patients take off their stockings. In more advanced stages, the skin exhibits a criss-crossed pattern of superficial cracks and fissures of the horny layer and appears pink to light red in color. The skin also becomes rough, and may develop an appearance similar to ichthiosis vulgaris (“pseudoichthiosis”). In more advanced stages of asteatotic eczema, dull erythema as well as oozing, crusting, abundant scratch marks, and disseminated nummular lesions are frequently seen. Vesiculation and lichefication are not usually regular features except when irritant or allergic contact dermatitis is superimposed.

By routine histology, xerotic skin appears fairly normal except for a compact and slightly irregular stratum corneum. In addition, asteatotic eczema exhibits mild focal spongiosis, parakeratosis and a lymphocytic infiltrate with neutrophils.

Non-invasive techniques can be used as adjunctive methods for the assessment of skin function without the need for biopsy. Among these methods are profilometry, squametry, in vivo image analysis, twistometre, and skin impedance measurement.

Proper attention must be given to the care of xerosis in order to avoid relapses. This care can include regular use of emollients, especially urea- or lactic acid-containing preparations, use of bath oils, and the elimination of factors that aggravate dry skin. The traditional approach to caring from dry skin or xerosis has been the universal use of “moisturizers”. Moisturizers can be divided into two categories: cosmetic and therapeutic. Cosmetic moisturizers are designed to fragrance the skin and temporarily make it smooth to the touch. Moisturizers, however, do not put water back into the skin externally, nor do they get incorporated into the intercellular lipids. Moisturizers simply attempt to retard transepidermal water loss and create an optimal environment for restoration of the stratum corneum barrier. The optimal water content for the stratum corneum is above 10% (depending on the measurement technique employed) and moisturizers can function to raise the cutaneous water content through occlusion or humectancy.

Occlusive moisturizers prevent evaporative water loss to the environment by placing an oily substance on the skin surface through which water cannot penetrate. This replenishes the moisture in the stratum corneum by moving water from the lower viable epidermal and dermal layers. There are many different classes of chemicals that can function as occlusive moisturizers. For example, hydrocarbon oils and waxes (e.g. petrolatum, mineral oil, paraffin, squalene), silicone (e.g. cyclomethicone, dimethicone), vegetable oils (e.g. grape seed oil, soybean oil), animal oils (e.g. mink oil, emu oil), fatty acids (e.g. lanolin acid, stearic acid), fatty alcohols (e.g. lanolin alcohol, cetyl alcohol), polyhydric alcohols (e.g. propylene glycol), wax esters (e.g. lanolin, beeswax, stearyl stearate), vegetable waxes (e.g. caranuba wax, candelilla wax), phospholipids (e.g. lecithin), and sterols (e.g. cholesterol).

One of the most effective occlusive moisturizers is petrolatum because it reduces transepidermal water loss by about 99%. Paradoxically, total occlusion of the stratum corneum is undesirable, since transepidermal water loss is the cellular signal that initiates barrier repair and the resulting synthesis of intercellular lipids. Complete cessation of transepidermal water loss results in the retardation of barrier repair, allowing water loss to return to its pre-treatment level once the complete occlusion has been removed.

Other moisturizers useful for rehydrating the stratum corneum are humectants. Humectants are substances that attract moisture, such as glycerin, honey, sodium lactate, urea, propylene glycol, sorbitol, pyrrolidone carboxylic acid, gelatin, hyaluronic acid, and some vitamins and proteins. The body utilizes hyaluronic acid and other glycoaminoglycans (GAGs) in the dermis as biologic humectants to prevent desiccation of the skin. Humectants can only hydrate the skin from the environment when the ambient humidity exceeds about 70%. Consequently, rehydration of the stratum corneum generally occurs by water that is attracted from the deeper epidermal and dermal tissues.

Many moisturizers combine both occlusive and humectant moisturizing ingredients because water drawn by a humectant to a damaged stratum corneum barrier will be lost to the atmosphere unless trapped by an occlusive. Humectants also help to improve the smoothness of xerotic skin by inducing corneocyte swelling and minimizing voids between the desquamating corneocytes.

Remoisturization of the skin should occur in four steps: 1) initiation of barrier repair by synthesis of intercellular lipids; 2) alteration of the surface cutaneous moisture partition coefficient; 3) onset of dermal-epidermal moisture diffusion; and 4) enhancement of the biologic humectant function by synthesis of extrafibrillar GAGs and proteoglycans. Remoisturization is facilitated by occlusion, but humectants should also be used to bind the water made available from the natural dermal reservoir. Among humectants frequently used in moisturizing products are propylene glycol, urea, and hyaluronic acid.

Propylene glycol is a sweet, viscous fluid that is soluble in water. It is used as a keratolytic agent at a concentration ranging from about 10 to about 20%. At higher concentrations, irritation is significant. Propylene glycol is also employed as a preservative and a penetration enhancer.

Urea is also soluble in water and alcohol, and has marked hydrating properties. Indeed, urea attracts and holds water, resulting in transepidermal water migration. Although this increases hydration of the stratum corneum, such hydration may come at the expense of epidermal water content when used topically as water evaporates from the stratum corneum and overall hydration of the epidermis may decrease in the absence of occlusion. At a strength of about 40%, urea is proteolytic and able to solubilize and denature proteins. At a strength ranging from about 10 to about 20%, urea has antimicrobial properties. Due to its hydrating properties, urea is commonly used as a 10 to 20% O/W (oil in water) cream for the treatment of dry skin and ichthyosis. Formulation in a greasy vehicle may cause a burning sensation. Urea is also very useful at 40% for the treatment of palmoplantar keratoderma and, under occlusion for chemical avulsion of the nails.

In addition, replacement of lipids normally present in the stratum corneum is also important. Some non-limiting examples of products useful for normalizing the structure and function of xerotic, aging skin include cholesterol sulfate, free sterols, free fatty acids, triglycerides, sterol wax/esters, squalene and n-alkanes.

Cholesterol sulfates make up only about 2 to 3% of the total epidermal lipids, but are important in corneocyte desquamation. Corneocyte desquamation appears to be mediated through the desulfation of the cholesterol sulfate.

Fatty acids are also important since the barrier function can be restored by topical or systemic administration of linoleic acid-rich oils in essential fatty acid-deficient animals.

Linoleic acid is an omega-6 fatty acid which the body cannot synthesize. The essential fatty acids (EFAs), therefore, must be obtained from the diet or other exogenous source.

An as-yet unmet challenge has been the efficient and appropriate maintenance and/or retention of physiologically correct amounts of epidermal/stratum corneum hydration at the onset of skin xerosis in combination with “relative occlusion” therapy. “Occlusion therapy” refers to the use of compounds that prevent evaporative water loss to the environment by placement of an oily substance on the skin through which water cannot penetrate. “Absolute occlusion therapy,” however, might refer to a retardation of barrier layer repair as it interferes with the biologic signal initiating replenishment of the intercellular lipid barrier.

With “relative occlusion therapy,” and in the presence of predetermined factors and conditions, stratum corneum hydration should be replenished by means of moving water anteriorly from the extra-fibrillar matrix of water-binding proteoglycans and glycosaminoglycans (GAGs). This is, of course, the normal physiological pathway for epidermal hydration. The body utilizes hyaluronic acid and other glycoaminoglycans (GAGs) in the dermis as biologic humectants to prevent desiccation of the skin, but the destruction of the epidermal barrier to the loss of water compromises this function.

In addition to enhancing the physiologic dermal extrafibrillar humectant functions, the epidermal barrier function should be repaired to further prevent loss of stratum corneum hydration to the atmosphere. This can be accomplished by the initiation of barrier repair with the simultaneous addition of critical intercellular lipids lost by normal degradation caused by intrinsic and extrinsic aging, and by other factors. These intracellular lipids include sphingolipids, free sterols and free fatty acids, which play a key role in barrier function and which are essential to trap water, thus preventing excessive water loss. The major lipid (by weight) found in the stratum corneum is ceramide.

According to certain embodiments of the present invention, compositions and methods for the treatment of xerosis and aged (extrinsic and/or intrinsic) skin are provided. These compositions and methods are directed to the restoration of collagen cross-links and replenishing the ground substance of the skin.

One exemplary inventive treatment includes the topical application of a topical composition such as a cream, lotion, and/or ointment. These topical compositions may be used by itself as the sole treatment. Alternatively, the topical composition may be used in conjunction with other mechanical and/or radiation-based skin remodeling, such as microdermabrasion, laser and radio-frequency-based skin remodeling, and other modalities intended to induce collagenesis as a culmination of the fibrosis of the wound repair response to trauma. When used in conjunction with these methods, the topical compositions for skin rejuvenation should be used at least once daily, and more beneficially twice daily, beginning 2 to 3 days prior to the invasive procedure. The topical compositions should also be used up to 45 days following the procedure.

Combining the use of an inventive topical composition with non-ablative mechanical and/or radiation based treatment significantly reduces the amount of mechanical and/or radiation based treatment required to effect clinical improvement in the skin. Although such non-ablative treatment modalities have been thought to have low risk of cancer, these modalities have recently been shown to cause damage to DNA. In particular, elevations in p16 and PCNA have been observed following long-term treatment with these non-ablative, photothermal modalities. These non-ablative procedures used alone take several months to effect clinical improvement in the skin, making DNA damage a significant risk. Therefore, reducing the length of time of treatment with such modalities is particularly desirable.

In addition, reducing the amount of time of treatment with non-ablative modalities markedly reduces costs of doing business for dermatologists and other treatment professionals. Although treatment of the skin with non-ablative processes typically requires numerous treatments over several months, treatment professionals are generally only able to charge a single fee for the entire treatment. Combining these treatment modalities with the inventive topical compositions reduces the number of treatments with the non-ablative modalities, and provides a continuous source of revenue from the repeat sales of the compositions.

According to one embodiment of this invention, topical compositions for the reversal of xerosis and/or aging are provided. One such topical composition may include a composition adapted to promote collagen and proteoglycan biosynthesis. In one embodiment, for example, the composition adapted to promote collagen and proteoglycan biosynthesis includes α-lipoic acid. The composition adapted to promote collagen and proteoglycan biosynthesis may further include other antioxidants, cupric salts or peptides, and/or essential amino acids, including methionine and/or cysteine to enhance the dermal biologic humectant function. These compositions adapted to promote collagen and proteoglycan biosynthesis provide the dermal aqueous reservoir required for remoisturization.

In one exemplary embodiment, a topical composition for the treatment of xerosis or aging of the skin includes: methionine and cysteine; a mixture of essential amino acids including isoleucine, leucine, lysine, phenylalanine, threonine, tryptophan, valine, histidine, and arginine; at least one antioxidant; at least one cross-linking agent; and at least one metallic catalyst

Methionine and cysteine may be present in the composition in any quantities sufficient to accelerate restoration of the integrity and fullness of the skin. In one embodiment, for example, methionine is present in the composition at a concentration ranging from about 2% to about 35% by weight based on the total weight of the amino acids. In another embodiment, methionine is present in the composition at a concentration ranging from about 2% to about 4% by weight based on the total weight of the amino acids. In yet another embodiment, methionine is present at a concentration of about 3.28% by weight based on the total weight of the amino acids. In one embodiment, methionine makes up from about 0.0005% to about 0.002% by weight of the composition. In another embodiment, methionine makes up about 0.001% by weight of the composition.

Cysteine may be present in the composition at a concentration ranging from about 2% to about 75% by weight based on the total weight of the amino acids. In one embodiment, for example, cysteine is present in the composition at a concentration ranging from about 25% to about 75% by weight based on the total weight of the amino acids. In another embodiment, cysteine is present at a concentration of about 40% by weight based on the total weight of the amino acids. In one embodiment, cysteine makes up from about 0.01% by weight to about 0.4% by weight of the composition. In another embodiment, cysteine makes up about 0.2% by weight of the composition.

The mixture of essential amino acids may be present in the composition in any quantity sufficient to accelerate restoration of the integrity and fullness of the skin. In one embodiment, for example, the mixture of essential amino acids (not including methionine or cysteine) makes up from about 0.005% by weight to about 0.5% by weight of the composition. In another embodiment, the mixture of essential amino acids (not including methionine or cysteine) makes up from about 0.1% by weight to about 0.4% by weight of the composition. In still another embodiment, the mixture of essential amino acids (not including methionine or cysteine) makes up about 0.3% by weight of the composition.

The at least one antioxidant may be present in the composition in any quantity sufficient to accelerate restoration of the integrity and fullness of the skin. In one embodiment, the at least one antioxidant is selected from lipoic acid, lipoic acid derivatives and analogues, ascorbic acid, and ascorbic acid derivatives. In an exemplary embodiment, the antioxidant is lipoic acid or a lipoic acid derivative or analogue. Non-limiting examples of suitable lipoic acids or lipoic acid derivatives or analogues include lipoic acid, dihydrolipoic acid, lipoic acid esters, dihydrolipoic acid esters, lipoic acid amides, dihydrolipoic acid amides, salts of lipoic acid, and salts of dihydrolipoic acid. Lipoic acid, also known as α-lipoic acid, thioctic acid, 1,2-dithiolane-3-pentanoic acid, and 1,2-dithiolane-3-valeric acid, have structures generally represented by the following Formula:

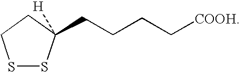

The disulfide (S—S) bond of lipoic acid is subject to reduction by chemical or biological reducing agents, leading to dihydrolipoic acid, in which the disulfide bond is replaced with two sulfhydryl (SH) groups. Because the two forms are readily interchangeable in vivo, both lipoic acid and dihydrolipoic acid, as well as their derivatives (such as esters, amides, and salts), can be used in the compositions according to the present invention.

When lipoic acid or a derivative or analogue of lipoic acid is used in the composition, it may be present at a concentration ranging from about 0.3% to about 2.0% by weight. In one embodiment, for example, it is present at a concentration ranging from about 0.5% to about 1.5% by weight. In another embodiment, it is present at a concentration of about 1.0% by weight.

According to another embodiment, the antioxidant is ascorbic acid or a derivative of ascorbic acid. The derivative of ascorbic acid may be a long-chain fatty acid ester of ascorbic acid selected from ascorbyl palmitate, ascorbyl myristate, and ascorbyl stearate. In one embodiment, the long-chain fatty acid ester of ascorbic acid is ascorbyl palmitate. According to one embodiment, the long-chain fatty acid ester of ascorbic acid is present in the composition at a concentration ranging from about 0.1% by weight to about 0.6% by weight. In another embodiment, the long-chain fatty acid ester of ascorbic acid (e.g. ascorbyl palmitate) is present in the composition at a concentration of about 0.3% by weight.

Other antioxidants may also be used. For example, the antioxidant may be a constituent of ginkgo, such as one selected from ginkgolide A, ginkgolide B, ginkgolide C, and bilobalide. In another alternative, the antioxidant may be an isoflavone, such as one selected from genistein, genistin, 6″-0-malonylgenistin, 6″-0-acetylgenistin, daidzein, daidzin, 6″-0-malonyldaidzin, 6″-0-acetylgenistin, glycitein, glycitin, 6″-0-malonylglycitin, and 6-0-acetylglycitin. In one embodiment, for example, the isoflavone is genistein or daidzein. Isoflavones can be isolated from soy or other phytochemical sources.

The metallic catalyst may be present in the composition in any quantity sufficient to accelerate restoration of the integrity and fullness of the skin. In one embodiment, the metallic catalyst is copper, in either its cuprous, cupric ionic or peptide complex form. In one exemplary embodiment, the copper is present in its cupric (Cu (II) ionic) form, as that is the form used by the enzyme lysyl oxidase. However, the body can readily inter-convert the various ionic forms of copper between the Cu(I) or Cu(II) forms. The metallic catalyst may be in the form of a copper salt, such as cupric acetate, cuprous acetate, cuprous chloride, cupric chloride, cuprous sulfate, cupric sulfate, or any other readily soluble copper salt or peptide complex. In one embodiment, the copper salt or peptide complex is present in a concentration ranging from about 1.0% to about 5.0% by weight. In another embodiment, the copper salt or peptide complex is present in a concentration ranging from about 1.5% to about 2.5% by weight. In yet another embodiment, the copper salt or peptide complex is present in a concentration of about 2.0% by weight.

In one embodiment, the mixture of essential amino acids (other than cysteine or methionine) may include: from about 5% to about 20% leucine; from about 10% to about 25% lysine; from about 5% to about 20% phenylalanine; from about 5% to about 25% threonine; from about 5% to about 20% tryptophan; from about 10% to about 25% valine; from about 5% to about 20% histidine; and from about 5% to about 20% arginine. In another embodiment, the mixture of essential amino acids (not including cysteine or methionine) includes: about 11.29% leucine; about 14.68% lysine; about 8.48% phenylalanine; about 20.91% threonine; about 7.91% tryptophan; about 16.94% valine; about 8.48% histidine; and about 11.29% arginine. In one embodiment, isoleucine is eliminated and the concentration of threonine is increased to about 20.91%. Threonine is used by cells to naturally create isoleucine according to the following pathway: Threonine→alpha-ketobutyrate (by action of threonine dehydratase enzyme)→alpha-aceto-alpha-hydroxybutyrate (by action of acetolactate synthase enzyme)→alpha,beta-dihydroxy-beta-methylvalerate (by the action of keto-acid reductoisomerase enzyme→alpha-keto-beta-methylvalerate (by the action of dihydroxyacid dehydralase enzyme)→isoleucine (by the action of transaminase enzymes).

The cross-linking agent may be present in the composition in any quantity sufficient to accelerate restoration of the integrity and fullness of the skin. In one embodiment, the cross-linking agent is a bioflavonoid selected from quercetin, quercitrin, kaempferol, kaempferol 3-rutinoside, 3′-methoxy kaempferol 3-rutinoside, 5,8,4′-trihydroxyl-6,7-dimethoxyflavone, catechin, epicachetin, epicachetin gallate, epigallocachetin gallate, hesperidin, naringin, rutin, vixetin, proanthocyanidin, apigenin, myricetin, tricetin, quercetin, naringin, kaernpferol, luteolin, biflavonyl, silybin, silydianin, and silychristin, and derivatives and glycosides of these compounds. In one embodiment, for example, the bioflavonoid is proanthocyanidin. Proanthocyanidin, whether in its monomeric, dimeric, or polymeric form, is an effective cross-linker of collagen and acts substantially without toxicity, as evidenced by Experimental Example 3, below. “Proanthocyanidin,” as used herein, refers to any and all of the monomeric, dimeric, and polymeric forms unless otherwise specified.

### EXPERIMENTAL EXAMPLE 3

### Effect of Proanthocyanidin on Collagen Stability

A. Cytotoxicity

NIH 3T3 cells were used in these studies. Cells were cultured in 24-well plates at a density of 5×10⁶ cells/well in 10% FBS/DMEM overnight. The medium was then replaced with complete medium supplemented with proanthocyanidin (MegaNatural, provided by Polyphenolics (Madera, Calif.)), in concentrations of 0, 20, 100, or 200 μg/mL, or glutaraldehyde (GA) in concentrations of 0, 0.1, 0.5, 1.0, or 5.0 μg/mL. Cells were incubated for 72 hours before cell counting and morphological studies.

B. Fixation Process

Fresh bovine tendon, pericardium strips, and processed collagen sponges (prepared with bovine tendon atelopeptide-collagen) were fixed with either 0.5% proanthocyanidin PBS solution (pH 7.4) or 0.625% GA/PBS solution for 48 hours at room temperature.

C. In Vitro Enzymatic Degradation

Proanthocyanidin-fixed tendon tissue together with fresh controls were digested with 0.2% collagenase (Worthington Biochemicals, NJ), at pH 7.4 for 24 hours at 37° C. Tissue integrity was checked at the end of the incubation using a standard histological method (hematoxylin-eosin (H&E)). To quantitate enzyme digestion rate, 500 mg of both Type I collagen sponges treated with proanthocyanidin or untreated were digested with 15 mL of 0.2% collagenase in PBS solution at 37° C. At predetermined intervals, 1.0 mL of solution was taken out and filtered through a 0.45-μM cellulose filter to separate solubilized collagen from insolubilized matrix. The amount of solubilized collagen was determined after total acid hydrolysis in 6N HCl for 24 hours at 100° C. by measuring hydroxyproline. The results are expressed as a percentage of the total collagen solubilized.

D. Melting Temperature Measurement

Melting temperature has been extensively used as an indicator of the amount of cross-linking in biopolymers. The fixed tissues and fresh tissues were assayed for their melting temperature by heating tissue strips (1×2 cm², n=3). The melting temperature was recorded when tissues started to shrink.

E. The Stability of Proanthocyanidin-Treated Tissue

After 48-hours of fixation in proanthocyanidin, tissue was incubated in PBS containing 0.5% sodium azide solution at 37° C. for preservation. For prolonged storage, tissues were kept in 40% ethanol/PBS (controls). After different time intervals, the shrinkage temperature of the tissues was measured after thorough rinsing.

F. In Vitro Cell Culture

Discs, 15 mm in diameter, were punched out from PA-treated bovine pericardium and inserted into the bottom of 24-well plates. After washing and equilibrating with PBS, human skin fibroblasts (8×10⁴/well, third passage kindly provided by Dr. Warren Garner, University of Southern California) were placed on top of the tissues. After 48 hours of culture in 10% FBS/DMEM, the medium was changed to a labeling medium ([³H]thymidine, 10 μCi/mL, 0.5% FBS/DMEM) followed by a 24 hour labeling period. Cell proliferation was assayed and collagen synthesis recorded as [³H]OH-proline incorporation in a culture medium containing 25 μg/mL ascorbic acid, 25 μg/mL β-APN and 25 μCi/mL [³H]proline in 0.5% FBS/DMEM after labeling for 48 hours.

G. Subcutaneous Implantation

Three-week-old Fischer 344 rats were obtained from Harlan Sprague Dawley, Inc. (Indianapolis, Ind.). NIH and University of Southern California IACUC guidelines for the care and use of laboratory animals were observed. Proanthocyanidin or GA-treated collagen sponges (1×1 cm²) and bovine pericardium (1×2 cm²) were implanted subcutaneously on the back of each animal (n=4). Similar materials without treatment were implanted as controls. The samples were retrieved after 3 and 6 weeks post-operation, and samples were processed for H&E and von Kossa staining, the latter to determine the extent of calcification.

H. Results

1. Cytotoxicity

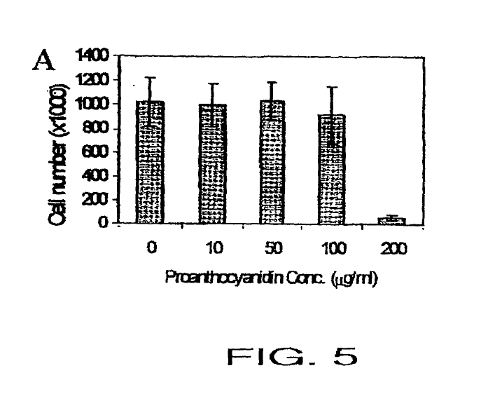

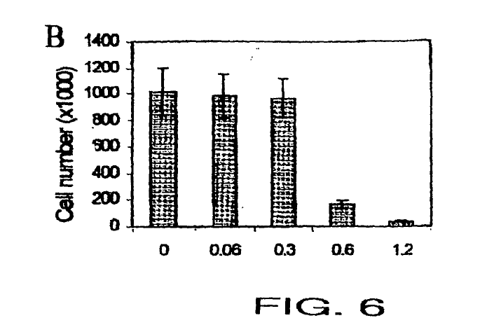

After a 72 hour incubation, cells grown in the medium supplemented with 0-100 μg/mL proanthocyanidin proliferated normally (See FIG. 5). No cytotoxicity of proanthocyanidin was observed until the concentration approached 200 μg/mL. On the other hand, GA exhibited obvious cytotoxicity, even at a concentration of 0.6 μg/mL (See FIG. 6). The potential cytotoxicity may arise from residues of unreacted or degraded cross-linking agents. In this Example, fibroblasts could grow with a high concentration of proanthocyanidin in the medium (200 μg/mL), whereas cells could not survive when the GA concentration was greater than 0.6 μg/mL. These results indicate that any polyphenolic residues, either from unreacted proanthocyanidin or from degradation of cross-linked materials, had little toxic effect.

2. Physicochemical Properties of Proanthocyanidin-Treated Tissue

When treated with 0.5% proanthocyanidin, tissues turned brownish in color because of the color of the solution. Table 2 (below) presents the melting temperature of different tissues treated with proanthocyanidin (PA) or GA. For both tendon and pericardium, melting temperatures increased dramatically in the proanthocyanidin group compared with fresh controls (p<0.05).

TABLE 2
Differences in Melting Temperatures of Fresh
and Treated Specimens Obtained from Two different
Sources (Tendon) and Pericardium)

| TISSUE | CONTROL | PA^{a} | GA |
|---|---|---|---|
| Tendon | 55 ± 0.5 | 80 ± 1.5 | 84 ± 1.0 |
| Pericardium | 70 ± 1.0 | 91 ± 2.0 | 94 ± 1.5 |

Specimens were treated in corresponding solutions for 48 hours (n = 3).
^{a}Proanthocyanidins, 0.5% in PBS.
^{b}Glutaraldehyde, 0.625% in PBS.

The increase in Tm from 66° C. to 86° C. of bovine pericardium upon reaction with proanthocyanidin indicates that effective cross-linking of collagen occurred under mild conditions. The cross-linking is likely to arise from hydrogen bonds formed between the polyphenolic structure of proanthocyanidin and collagen chains that are in their physiological triple helical conformation. Therefore, these findings are readily applicable to a broad range of collagens.

3. Concentration of Proanthocyanidin and Cross-linking Efficiency

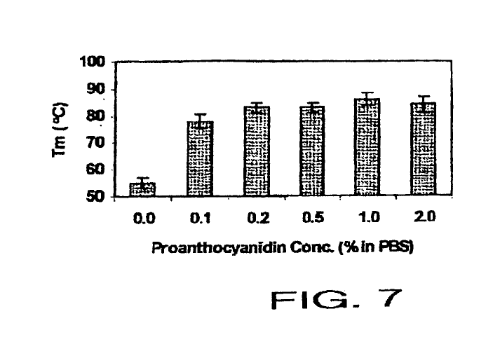

By comparing the concentration of proanthocyanidin with the melting temperatures (See FIG. 7), it was found that 0.5% was optimal for maximally cross-linking the tissue. However, the concentration of the cross-linking solution used is an important consideration not only for the degree of cross-linking, but also for cross-linking efficiency. When 1.0% proanthocyanidin was used to fix bovine tendon, the center of this rather large tendon was not fixed well and was readily digested by collagenase. It was found that lower concentrations of fixative penetrated into the tissues more readily, thus increasing the efficiency of fixation particularly when 0.05M Ca(OH)₂ was added to the fixation solution. Ca(OH)₂, a chaotropic agent, at this concentration, appears to help proanthocyanidin penetrate while keeping the tissue from swelling to any significant degree.

4. In Vitro Enzymatic Degradation

The histological appearance of fresh, proanthocyanidin-fixed (0.5% proanthocyanidin treated), and GA-fixed (0.625% GA treated) bovine pericardium, stained with H&E, after 24 hours of collagenase digestion were analyzed using H&E staining. In all instances, fresh tissues disintegrated into small pieces. In contrast, the collagen fibril structure of the GA- and proanthocyanidin-treated tissues remained intact.

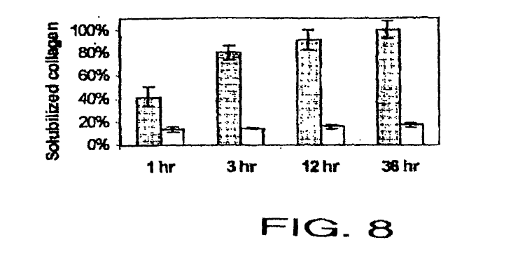

FIG. 8 illustrates the enzyme digestion rate by checking the amount of solubilized collagen at different digestion times when proanthocyanidin-treated collagen sponges and controls were digested (1 hour, 3 hours, 12 hours, 36 hours; open bar, untreated control; shaded bar, treated with proanthocyanidin). The solubilized collagen was quantitated by measuring hydroxyproline in solution. Fresh pericardium was completely digested after 36 hours, whereas proanthocyanidin-treated tissues remained intact after collagenase treatment.

5. Cell Proliferation and Collagen Synthesis on the Surface of Treated Pericardium Matrices

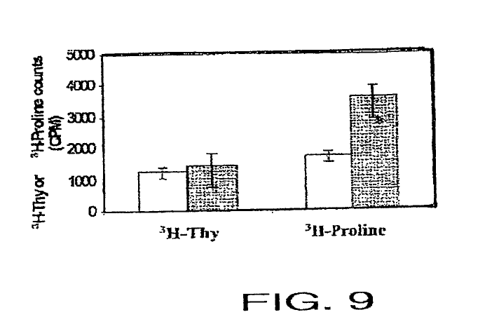

There are no significant differences in cell proliferation rates of human skin fibroblasts cultured on proanthocyanidin-treated or non-treated fresh bovine pericardium. On the other hand, proanthocyanidin treatment seems to enhance the cell's ability to deposit collagen (p<0.005; FIG. 9). In FIG. 9, cell proliferation rates and collagen synthesis of human fibroblasts cultured on proanthocyanidin-treated pericardium tissue (untreated, open bars; proanthocyanidin-treated, shaded bars). Cell proliferation rates were assayed by thymidine incorporation and collagen synthesis was assayed by hydroxyproline incorporation (n=5).

6. Stability of Proanthocyanidin-Treated Tissue

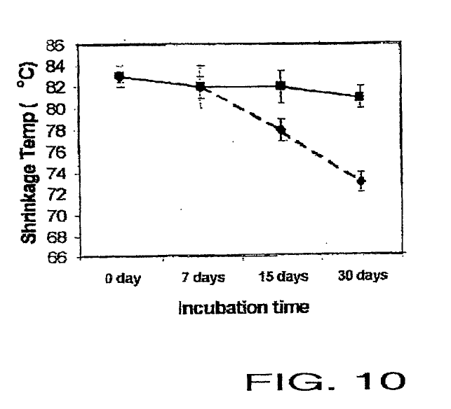

The stability of proanthocyanidin-induced cross-linking was evaluated under physiological conditions in vitro. When tissue was stored in PBS at 37° C. for 30 days, the hydrogen bonds were destabilized and shrinkage temperature began to decrease, but when the dielectric constant of the solution was lowered by adding 40% ethanol to the PBS, cross-links remained stable and the shrinkage temperature remained constant (See FIG. 10), reflecting the participation of hydrogen bonding in the process.

In FIG. 10, the shrinkage temperature in PBS is shown by the solid line; the shrinkage temperature in 40% ethanol/PBS is shown by the dashed line. Pericardium strips were treated with 0.5% proanthocyanidin for 24 hours before being stored in the different solutions. The storage temperature was 21° C.

7. Subcutaneous Implantation

One week postoperatively, untreated pericardium (controls) gave rise to a notable inflammatory reaction, whereas proanthocyanidin-treated specimens showed cell invasion and in-growth. Glutaraldehyde-treated samples, after being thoroughly rinsed, exhibited a lesser inflammatory reaction. Three weeks postoperatively, control tissues started to disaggregate, whereas the proanthocyanidin- and GA-fixed tissues retained their integrity. The proanthocyanidin treated specimen appears to be the most tissue compatible. New fibroblasts penetrated and proliferated inside the tissue.

Six weeks postoperatively, control tissue could not be retrieved because it had been completely degraded. On the other hand, the proanthocyanidin-treated specimens were just starting to degrade, whereas GA-treated tissues were still intact. Von Kossa staining, which specifically indicates the presence of calcification, showed that there was no calcification in proanthocyanidin-treated tissues, whereas GA-treated tissues exhibited dystrophic calcification (data not shown). In FIG. 17, tissues are shown at 1 and 3 weeks postoperatively; implants were retrieved at those time points (PA, proanthocyanidin; GA, glutaraldehyde; H&E staining; original magnification, ×40).

Collagen has been used extensively in the manufacturing of bioprostheses and in the design of tissue engineered scaffolds. Of course, as indicated above, there are many other circumstances in which the stability and maturation of collagen is of critical importance, such as in the present specification pertaining to skin repair and rejuvenation. Fixation of biological tissues can reduce their antigenicity and increase their resistance to enzymatic degradation after implantation. Various cross-linking reagents, which include formaldehyde, glutaraldehyde, epoxy compounds, and carbodiimide, have been used, but all have drawbacks, including toxicity, cross-linking rates that are difficult to control, and instability.

Proanthocyanidin (PA) compounds appear to crosslink collagen and assist in the maturation of collagen while retaining its stability. They are naturally occurring plant metabolites widely available in fruits, vegetables, nuts, seeds, flowers, and barks. Proanthocyanidins are part of a specific group of polyphenolic compounds and belong to the category known as condensed tannins.

Proanthocyanidins increase collagen synthesis and accelerate the conversion of soluble collagen to insoluble collagen during development. In skin fibroblast cultures derived from Marfan patients and those of patients with Ehler-Danlos Type V, the excessive solubility of collagen can be corrected by the addition of a synthetic proanthocyanidin to the culture medium. They also inhibit the catabolism of soluble collagen in animal studies, stimulate normal skin fibroblast production, and increase the synthesis of the extracellular matrix, including collagen and fibronectin. Proanthocyanidins are natural products with polyphenolic structures that have the potential to give rise to stable hydrogen bonded structures and generate non-biodegradable collagen matrices. Furthermore, proanthocyanidins are widely used as natural antioxidants and free-radical scavengers, and have proven to be safe in different clinical applications and as dietary supplements. They lack acute and sub-acute toxicity and have free-radical-scavenging abilities.

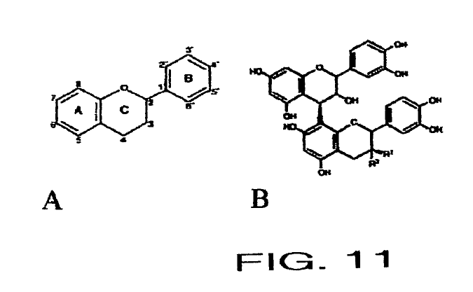

In proanthocyanidin, a benzene-pyran-phenolic acid molecular nucleus is the core structure of the oligomeric (See FIG. 11) and the polymeric forms of such a complex. In FIG. 11, (B) is the dimer form.

Four mechanisms for interaction between proanthocyanidin and proteins have been postulated, including covalent interactions, ionic interactions, hydrogen bonding interactions, and hydrophobic interactions. The interactions between proanthocyanidin and collagen can be disrupted by detergents or hydrogen-bond-weakening solvents, suggesting that proanthocyanidin and collagen complex formation involves primarily hydrogen bonding between the protein amide carbonyl and the phenolic hydroxyl. The relatively large stability of these cross-links compared with those between proteins and other phenols such as tannins suggests a structure specificity, which, although encouraging hydrogen bonding, also creates hydrophobic pockets. Such microenvironments, by virtue of decreasing the effective dielectric constant, enhance the stability of hydrogen bonds. Hydrogen bonds that are not stabilized by adjacent hydrophobic bonds can be dissociated by treatment with aqueous buffers. Alcohols, on the other hand, by decreasing the dielectric constant of the medium, also stimulate proanthocyanidin-collagen interactions. Therefore, in the experiments reported in Experimental Example 3, above, the cross-linked matrices were maintained in a 40% alcohol solution for long-term storage.

Proline-rich proteins like collagen have an extremely high affinity for proanthocyanidin. Proline, an imino acid with a carbonyl oxygen adjacent to a secondary amine nitrogen, is a very good hydrogen bond acceptor. Therefore, proline-rich proteins like collagen form especially strong hydrogen bonds with proanthocyanidin. Because collagen is a helical structure, as outlined above, accessibility of the peptide backbone is enhanced for the purpose of hydrogen bonding. Hydrogen bond formation, by stabilizing the collagen fibers, is responsible for the increase in the denaturation temperature of the fixed tissue. The shrinkage temperature (denaturation temperature) of the fixed bovine pericardium increased from 66° C. to 86° C., thereby demonstrating the efficacy of the cross-linking by proanthocyanidin.

Chronic cytotoxicity is always of primary concern when agents that penetrate the skin are being evaluated for effectiveness. Proanthocyanidins are widely used as food supplements, and their lack of toxicity has been extensively demonstrated. In addition, proanthocyanidins possess antibacterial, antiviral, anti-carcinogenic, anti-inflammatory, and anti-allergic activities. Proanthocyanidin is about 120 times less toxic than glutaraldehyde, a currently used tissue stabilizer. As shown by Experimental Example 3, fixed tissue is resistant to digestion by bacterial collagenase. After subcutaneous implantation for periods ranging from 3 and 6 weeks, no apparent degradation of the glutaraldehyde- or proanthocyanidin-fixed tissue was observed, whereas fixed tissue rapidly disintegrated. More fibroblasts migrated and proliferated inside the proanthocyanidin-fixed implants compared with GA-fixed implants. Tissues cross-linked with proanthocyanidin manifested an enhanced collagen expression and deposition and did not calcify after implantation. Fibroblasts cultured in the presence of proanthocyanidin increased their rate of collagen synthesis. GA, on the other hand, even after thorough rinsing, continued to be cytotoxic, inhibit collagen synthesis, and encouraged dystrophic calcification.

The results reported in Experimental Example 3 demonstrate the feasibility of using proanthocyanidin to crosslink collagen in the skin as part of a method for reversing damage to the skin, such as solar damage, incisional trauma or the effects of aging. These results demonstrate that proanthocyanidin is an effective cross-linker of collagen that promotes collagen stability and maturation.

In another alternative embodiment, the cross-linking agent may be a flavonoid that is a component of silymarin. Silymarin is an extract of the milk thistle plant, Silybium marianum. Milk thistle belongs to the aster family (Asteraceae or Compositae), which includes daisies, thistles, and artichokes. Silymarin consists of a mixture of three flavonoids that are found in the fruit, seeds, and leaves of the milk thistle plant: silybin (silybinin), silydianin, and silychristin. Silybin is the main component and is thought to have the most biological activity.

When the cross-linking agent is proanthocyanidin, it may be present in the composition at a concentration ranging from about 0.3% to about 2.0% by weight. In one embodiment, for example, it is present in the composition at a concentration ranging from about 0.5% to about 1.5% by weight. In another embodiment, it is present in the composition at a concentration of about 1.0% by weight.

Similarly, when the cross-linking agent is silybin, it may be present in the composition at a concentration ranging from about 0.3% to about 2.0% by weight. In one embodiment, for example, it is present in the composition at a concentration ranging from about 0.5% to about 1.5% by weight. In yet another embodiment, it is present in the composition at a concentration of about 1.0% by weight.

Other cross-linking agents are known and can also be used. For example, the protein decorin, which interacts with collagen, can be used as a cross-linking agent. Decorin is a member of the leucine-rich repeat (LRR) protein family and includes a 36.5-kDa core protein substituted with one glycosaminoglycan chain on an amino-terminal Ser-Gly site. The core protein contains ten leucine-rich repeats flanked by disulfide bond stabilized loops on both sides. It contains additional sites for glycosylation (N-linked glycosylation sites) within the leucine-rich repeats. The glycosaminoglycan chain backbone includes repeating disaccharide units of N-acetylgalactosamine and glucuronic acid, the latter often being converted into iduronic acid through epimerization at carbon 5. As the chains are elongated, they are modified by sulfation, resulting in chondroitin sulfate and dermatan sulfate, respectively. The degree of epimerization and sulfation varies between tissues. Decorin can also exist without glycosaminoglycan substitutions or with two glycosaminoglycan substitutions. Decorin interacts with collagen via its core protein and influences collagen fibrillogenesis. In addition, decorin decorates the surface of collagen fibers at the d and e bands, hence the name decorin. Decorin interacts with fibrillar collagens and affects the fibril diameter in vitro resulting in thinner fibrils. The interaction is mainly via the leucine-rich repeats 4-5 of the decorin core protein. In addition to the fibrillar collagens I, II, III, and V, decorin also interacts with collagens VI, XII, and XIV. Accordingly, decorin can be used as a cross-linking agent. Moreover, decorin has anti-inflammatory and anti-fibrotic properties because of its interaction with transforming growth factor-β (TGF-β), as well as its interaction with other proteins such as fibronectin, thrombospondin, the complement component C1q, and epidermal growth factor receptor EGFR. Still other protein cross-linking agents exist and can also be used.

According to another embodiment, a topical composition for the reversal of xerosis and/or aging includes a composition adapted to promote the biosynthesis of collagen, elastin and proteoglycans, a transdermal delivery agent or composition for transdermally delivering the composition through the skin, and a pharmaceutically acceptable carrier. Any suitable transdermal delivery agent or composition may be used, and in one embodiment is as described above. Similarly, the pharmaceutically acceptable carrier may be any suitable such carrier, and in one embodiment is as described above.

An alternative embodiment is directed to a method of topically replacing critical intercellular lipids in the form of sphingolipids, free sterols and free fatty acids, such as linoleic acid to rebuild the epidermal barrier function.

According to another embodiment, a method is provided for topically applying exogenous humectants such as urea (in a concentration of about 2% by weight), propylene glycol (in a concentration of about 2% by weight), and hyaluronic acid.

In still another embodiment, a method is provided for reversing xerosis by topically applying a “relative occlusive” agent such as propylene glycol (in a weight percent of about 2%) or other occlusive agent, such as lecithin or cholesterol. Such occlusive agents “relatively” retard transepidermal water loss but not the “cellular signal” required to restore the epidermal barrier layer.

According to yet another embodiment of the invention, emollients are provided for the care of skin suffering from xerosis, such as urea-containing preparations.

The inventive topical compositions described herein may be made by any suitable method, including standard methods used to make cosmetic preparations and pharmaceutical compositions intended for application on the skin. Non-limiting examples of suitable procedures include mixing techniques (both manual and mechanical mixing), homogenization mixing and sweep mixing. The mixing techniques can be chosen based on variables such as the viscosity of the components to be mixed and the volume of those components, as well as the relative proportion of lipid-soluble and water-soluble ingredients. The individual active ingredients may be added sequentially, and benzyl alcohol and/or other transepidermal delivery agents are added to the desired final concentration. Water and oil phases are heated separately to 70° C., blended, and cooled with normal mixing.

The inventive topical compositions described herein can be applied by users once or more daily, depending on age, skin condition, and other variables readily apparent to the user. In one embodiment, however, the composition is applied topically twice daily, and may be applied in the evening after removal of makeup and cleansing of the skin.

According to another embodiment of the invention, a method of repairing damage to the skin includes applying a topical composition according to an embodiment of the present invention to the skin in a quantity effective to repair damage to the skin. The damage to the skin can result from solar or chronological aging, xerosis, dry skin or damage from other trauma, such as incision. The topical compositions according to certain embodiments of the invention may also be used to reduce the occurrence of rhytides.

In yet another embodiment of the invention, a method of promoting the cross-linking of dermal collagen includes applying a topical composition according to an embodiment of the present invention to the skin in a quantity effective to promote cross-linking of dermal collagen. Cross-linking of collagen imparts structural integrity, maturation, resistance of solubility, and enduring stability. Each of these characteristics are critical to the restoration and rejuvenation of aged or otherwise damaged skin.

In another embodiment of the invention, a method of treating xerosis or aging of the skin includes the application of an inventive topical composition in a single comprehensive treatment in which non-penetrating agents are retained on the epidermal surface while penetrating agents are delivered transdermally. Alternatively, treatment is accomplished by two sequential applications in which the penetrant(s) and agents selected for transdermal delivery (e.g. those of appropriate size for transdermal delivery) are applied in a first treatment, while the remaining agents (such as occlusive agents, essential fatty acids, and emollients) are applied in a second treatment. One exemplary two-stage treatment includes a first, diurnal application of an occlusive agent, lipid replacement agent, and/or emollient, and a second, nocturnal application of transdermally delivered agents such as collagen biosynthesis agents and other transdermally delivered agents.

Whichever treatment method is used, to be effective in the reversal of solar aging and/or xerosis, the agents for enhancing collagen and proteoglycan biosynthesis must penetrate the epidermis to be bio-available to the dermis. The transdermal delivery of a topical composition for the treatment of aging, xerosis or dry skin enhances the biosynthesis of collagen, elastin and proteoglycans. The combination of a topical composition for the treatment of aging, xerosis or dry skin with two or more transdermal penetrants working synergistically enables transport of the topical composition into the dermis within about 30 minutes from the time of topical administration. Once available, fibroblastic activity occurs within about 30 minutes from the time of topical administration, demonstrating the efficacy of the compositions and methods of this invention.

The inventive transdermal delivery compositions discussed above can be used to deliver drugs or agents through the stratum corneum and epidermis to treat skin damaged by aging (either intrinsic or extrinsic), xerosis of the skin or dry skin. In one embodiment, the skin is first treated with a transdermal delivery composition and the target drug is thereafter administered through the permeabilized skin. Alternatively, the target drug or agent (e.g. the inventive topical composition described herein) and a transdermal delivery composition are combined in a single topical composition which is administered to the skin. In still other embodiments, the transdermal delivery composition is combined with another method for permeabilizing the stratum corneum and epidermis and the target drug or agent is thereafter applied to the permeabilized skin. In other alternatives, the topical composition including both a transdermal delivery composition and the target drug or agent is combined with another method for permeabilizing the stratum corneum and epidermis.

In one embodiment, the bioavailability of the topical compositions for the treatment of xerosis or aging of the skin is facilitated by the novel transdermal delivery methods and compositions described above. Upon transdermal delivery of the topical treatment composition, biosynthesis of the extracellular matrix, including collagen and elastin connective tissue and their supportive extrafibrillar proteoglycan ground substance, is enhanced. The water binding capability within the dermis (i.e. the dermal reservoir) is also enhanced.

Those agents not intended for transdermal transport are absorbed superficially and subsequently rebuild the epidermal permeability barrier. These agents may include fatty acids and relative occlusive agents and generate a water-tight outer skin layer (the stratum corneum) which protects the organism from desiccation due to excessive transcutaneous water loss. As such, the inventive methods and compositions enhance the dermal water reservoir while also restricting transcutaneous water loss.

The treatment of aged skin by the inventive methods and compositions induces rapid deposition of new collagen, elastin and proteoglycans, and naturally reverses both extrinsic (solar) and intrinsic (chronological) aging effects. In particular, the newly deposited collagen creates a new and more expanded fibrous network, and together with the deposition of proteoglycans (which hold water and prevent xerosis) in the inter-fibrillar spaces, greatly improves the texture of aging skin. The result is an epidermal layer having enhanced thickness, which improves skin texture and appearance.

### IV. Retinoids and Skin Lightening Agents for the Treatment of Photo-Aging

According to some embodiments of the present invention, topical compositions are provided for the treatment of photo-aging or other skin damage that include a retinoid or retinoid analogue, a skin lightener or both a retinoid and a skin lightener. Photo-aged skin is often characterized by the appearance of fine and coarse wrinkling, rough texture, sallow color and irregular pigmentation. Photo-aging is the consequence of UV-induced damage to the skin and is characterized by reduced expression of RXR-α and RAR-γ (the two major nuclear receptors in keratocytes) in the acute setting, and by up-regulation of AP1-driven matrix metalloproteinases.

Photo-aging and/or other damage may also cause irregularities in skin pigmentation. Skin pigmentation disorders are rather common and widespread, leading to a large demand for skin lightening products. The color of normal human skin is due primarily to melanin, hemoglobin, and carotenoids. Of these pigments, melanin is of particular importance in skin pigmentation and cosmetology. Skin color is largely determined by the type and amount of melanin synthesized by melanocytes (melanin-producing skin cells) and its distribution pattern in the surrounding keratinocytes. Two types of melanin are present in human skin: eumelanin and pheomelanin. Eumelanin is the dark brown pigment found in most skin, hair and eyes, and its production is stimulated by exposure to ultraviolet light, Pheomelanin is a yellow-orange pigment found mainly in the skin of very fair-skinned people, particularly those with red hair.

Melanin forms through a series of oxidative reactions involving the amino acid tyrosine in the presence of the enzyme tyrosinase. Tyrosinase converts tyrosine to dihydroxyphenylalanine (DOPA) and then to dopaquinone. Subsequently, dopaquinone is converted to dopachrome through auto-oxidation, and finally to dihydroxyindole or dihydroxyindole-2-carboxylic acid (DHICA), which polymerize to form eumelanin. The latter reactions occur in the presence of dopachrome tautomerase and DHICA oxidase. In the presence of cysteine or glutathionine, dopaquinone is converted to cysteinyl DOPA or glutathione DOPA; subsequently, pheomelanin is formed.

Skin hyperpigmentation (or melasma) has a variety of causes, including exposure to UV light, genetic makeup, wounds, age (e.g. “age spots”), pregnancy (e.g. “mask of pregnancy or “chloasma”), oral contraceptive use, exposure to certain chemicals, a number of skin and systemic diseases, and other factors. A safe and effective topical skin-lightening formulation can be useful for treating such localized epidermal hyperpigmentation. Such a formulation may also be useful to mask areas of skin hypopigmentation, such as in vitiligo or trauma-induced hypopigmentation, by lightening the surrounding skin.

Hydroquinone is widely used as a skin-lightening agent. However, the prolonged topical use of this compound has been associated with a variety of disorders including diabetes, hypertension, ochronsis, periorbitary dyschromia, infectious dernatosis, contact eczema, extended dermatophytosis and necrotizing cellulites. Hydroquinone has also shown genotoxic and mutagenic activities. Reports of toxicity have led to the banning of hydroquinone in Europe for use as a depigmenting agent and in the U.S. its use is limited to solutions having concentrations of 2% or lower.

Other depigmenting agents include kojic acid, which has moderate depigmenting activity but is commonly irritating. Azelaic acid and certain hydroxy acids, for example glycolic acid, have similar properties. The prenylated flavonol artocarpin has shown some efficacy for skin-lightening following ultraviolet-induced skin pigmentation.

Localized skin hyperpigmentation may be treated by topical drug administration. Such administration restricts the treatment to hyperpigmented areas leaves normal skin unaffected by the drug. Localized topical administration may also help avoid incurring high systemic drug levels and resulting toxicity or other adverse effects.

Topical retinoids, including tretinoin, improve fine and coarse wrinkling and lighten uneven pigmentation. Topical retinoids hypothetically promote cellular de-differentiation and extra-cellular matrix synthesis by restoring nuclear retinoid receptors and inhibiting AP1 activity. Histologic findings after repeated topical application include: compaction of the stratum corneum, epidermal hyperplasia (acanthosis), correction of atypica (e.g. actinic keratoses), dispersion of melanin granules, increased dermal collagen synthesis, and angiogenesis. Physical improvements to the skin include smoother skin, rosy glow, decreases in blotchy pigmentation, and diminished fine lines and wrinkles.

Vitamin A (retinol) and related compounds with either structural (retinol derivatives) or functional (vitamin activity) analogy are known as retinoids. All-trans-retinoic acid (tretinoin or at-RA) is a naturally occurring metabolite of retinol.

Retinoids have nuclear receptors known as retinoic acid receptors (RARs) and retinoid X receptors (RXRs). New retinoids are being developed focusing on binding properties to specific retinoid receptors. Topical retinol and retinal (retinoic acid precursors) are included in cosmeceutical preparations because they induce less irritation than topical tretinoin or isotretinoin. However, the efficacy of retinol and retinal is still unknown.

As noted above, retinoids or retinoid compounds or derivatives (collectively referred to herein as “retinoids”) are Vitamin A derivatives. They are used in topical compositions to treat a variety of sin conditions, including acne, actinic damage, dandruff, eczema, fine lines, psoriasis, warts and wrinkles. Some retinoids that have been used include isotretinoin, retinal, retinol, retinoic acid, retinyl acetate, retinyl palmitate, retinyl propionate, synthetic retinoid mimics, and tretinoin. The amount of retinoid used in a topical composition varies depending on the condition to be treated as well as on the composition and the retinoids used.

It generally takes three to six months of daily applications to see clinical improvement. Frequent cutaneous irritation is the limiting factor with tretinoin treatment. Retinaldehyde is as effective as tretinoin in treating photo-damage, but has a better tolerance profile. It is believed that the reduction in side effects seen with retinaldehyde (compared with retinoic acid) is due to a more controlled delivery of retinoic acid to target cells, thus limiting an overload of retinoic acid in the skin, which may be partly responsible for cutaneous irritation.

Regardless of the preparation or indication, the most important element in retinoid therapy is patient education. Local skin irritation characterized by erythema and peeling can be expected, and noticeable beneficial effects may take weeks or months to appear. Administration of topical retinoids should be tailored depending upon cutaneous irritant reactions, which may mean decreasing the concentration or frequency of application. Generally, tretinoin cream is administered at a concentration of 0.02% by weight in an oil-in-water emulsion. After a single administration of 0.05% tretinoin, as well as after repeated daily applications for 28 days, absorption may be less than 2% and the bioavailability was even less after repeated applications. Based on this reality, there may be a tendency to over-treat in order to obtain clinical improvement. However, one treatment method has been to begin with a lower concentration formulation, and increase the concentration as tolerance increases. Another tactic is to stagger the applications, for example, by applying a given concentration every other day.

Topical retinoid regimens also typically include the application of daytime moisturizers with sunscreen. The use of daytime moisturizers may help minimize the cutaneous irritation caused by the retinoid treatment. The sunscreen helps prevent further photo-damage to the skin, thereby aiding the retinoid treatment to repair the damage. The use of moisturizers and sunscreens, therefore, increase patient compliance.

Retinoids can induce both direct and indirect effects on gene transcription. The direct effects are mediated through binding to their hormone response element (retinoid hormone response element (RARE) in the promoter region of target genes whose transcription is activated. It is probable that many of the differentiating-inducing actions are mediated by this mechanism. In contrast, the indirect effects of retinoids result from the down regulation of genes that do not contain a RARE in their promoter region. The retinoid-receptor complex probably antagonizes various transcription factors such as AP1 or NF-IL6 by competing for commonly required co-activator proteins, thereby down regulating expression of AP1 and NF-IL6 responsive genes. The anti-proliferative and anti-inflammatory actions of retinoids are believed to be mediated by this type of negative, indirect gene regulatory mechanism. AP1 and NF-IL6 are key transcription factors in proliferative and inflammatory responses and “dissociating retinoids” have been synthesized that possess indirect anti-AP1 function but not direct gene transactivation function.

According to certain embodiments of the present invention, topical compositions for the treatment of photo-aging, photo-damage and/or hyperpigmentation include a retinoid, such as tretinoin in combination with an effective transdermal delivery system, agent or composition. As noted above, the transdermal delivery agent may be applied to the skin first followed by application of the topical composition including the retinoid. Alternatively, the topical composition may include the retinoid and the transdermal delivery agent in a single topical composition. In addition, either one of these treatment methods may be combined with other methods for increasing the permeability of the skin.

In one embodiment, a topical composition for the treatment of hyperpigmentation includes a skin lightening agent, a skin penetrant and a topical pharmaceutically acceptable carrier. The topical composition may have a pH ranging from about 3.0 to about 7.4. The skin penetrant may be any suitable skin penetrant, but in one embodiment is a transdermal delivery agent or composition as described above. The topical pharmaceutically acceptable carrier may also be any suitable carrier, but in one embodiment is a topical pharmaceutically acceptable carrier as described above.

The skin lightening agent may be any suitable agent capable of lightening hyperpigmented areas of the skin. Non-limiting examples of suitable skin lightening agents include hydroquinone, kojic acid, azelaic acid, glycolic acid and artocarpin. In one embodiment, for example, the skin lightening agent is hydroquinone. In another embodiment, the skin lightening agent is a hydroquinone derivative selected from 4-[(tetrahydro-2H-pyran-2-yl)oxy]phenol and 4-[(tetrahydro-2H-thiopyran-2-yl)oxy]phenol. Other skin lightening agents are known and can also be used.

The skin lightening agent may be present in the topical composition in an amount ranging from about 1.5% by weight to about 4.0% by weight. In one embodiment, the skin lightening agent is present in an amount ranging from about 1.8% to about 2.2% by weight. In another embodiment, the skin lightening agent is present in an amount of about 2.0% by weight.

According to another embodiment of the present invention, a topical composition for the treatment of skin includes a retinoid, a skin penetrant and a topical pharmaceutically acceptable carrier. The topical composition may have a pH ranging from about 3.0 to about 7.4. The penetrant may be any suitable penetrant, and in one embodiment may be a transdermal delivery agent or composition as described above. Similarly, the topical pharmaceutically acceptable carrier may be any suitable carrier, and in one embodiment includes a topical pharmaceutically acceptable carrier as described above. However, when the retinoid used is tretinoin (which is a weak organic acid), the topical pharmaceutically acceptable carrier need not include an additional acid to adjust the pH.

Non-limiting examples of suitable retinoids include isotretinoin, retinal, retinol, retinoic acid, retinyl acetate, retinyl palmitate, retinyl propionate, synthetic retinoid mimics, and tretinoin. In one embodiment, the retinoid is tretinoin. The retinoid may be present in the composition in an amount ranging from about 0.005% to about 1.0% by weight of the composition. In one embodiment, for example, the retinoid is present in an amount ranging from about 0.10% to about 0.75% by weight. In another embodiment, the retinoid is present in an amount of about 0.50% by weight.

In yet another embodiment of the present invention, a topical composition for the treatment of skin includes a retinoid, a skin lightener, a skin penetrant and a topical pharmaceutically acceptable carrier. The topical composition may have a pH ranging from about 3.0 to about 7.4. The penetrant may be any suitable penetrant, and in one embodiment may be a transdermal delivery agent or composition as described above. Similarly, the topical pharmaceutically acceptable carrier may be any suitable carrier, and in one embodiment includes a topical pharmaceutically acceptable carrier as described above. However, as noted above, when the retinoid is tretinoin (which is a weak organic acid), the topical pharmaceutically acceptable carrier need not include an additional acid to adjust the pH.

The retinoid and skin lightener are as described above. The amounts of these ingredients are the same as described above.

The inventive topical compositions described herein may be made by any suitable method, including standard methods used to make cosmetic preparations and pharmaceutical compositions intended for application on the skin. Non-limiting examples of suitable procedures include mixing techniques (both manual and mechanical mixing), homogenization mixing and sweep mixing. The mixing techniques can be chosen based on variables such as the viscosity of the components to be mixed and the volume of those components, as well as the relative proportion of lipid-soluble and water-soluble ingredients. The individual active ingredients may be added sequentially, and benzyl alcohol and/or other transepidermal delivery agents are added to the desired final concentration. Water and oil phases are heated separately to 70° C., blended, and cooled with normal mixing.

According to other embodiments of the present invention, methods of using topical compositions for the treatment of skin are provided. Topical compositions including retinoids may be used to treat skin conditions including acne, actinic damage, dandruff, eczema, fine lines, psoriasis, warts and wrinkles. To treat one of these conditions, an effective amount of a topical composition including a retinoid is applied to the skin in need of treatment.

Topical compositions including skin lighteners may be used to treat hyperpigmentation of the skin. To treat hyperpigmentation, an effective amount of a topical composition including a skin lightener is applied to the skin in need of treatment. The hyperpigmentation to be treated may be caused by UV light, genetic makeup, wound, age spots, chloasma, oral contraceptive use, chemical exposure or any other cause. Topical compositions include a skin lightener may also be used to mask an area of hypopigmentation by applying an effective amount of the topical composition to skin surrounding the hypopigrnented area. Hypopigmentation can be caused by vitiligo or trauma.

In one exemplary method of treatment, an inventive topical composition is applied over a period of time. Specifically, a safe and effective amount of the composition including the retinoid and/or the skin lightening agent is applied in increments ranging from about 1 g/cm² to about 10 g/cm² per application. In another embodiment, the composition is applied in increments ranging from about 2 g/cm² to about 8 g/cm² per application. In still another embodiment, the composition is applied in increments ranging from about 3 g/cm² to about 7 g/cm² per application. In still yet another embodiment, the composition is applied in increments ranging from about 4 g/cm² to about 5 g/cm² per application.

The composition may be applied from about twice a week to about four times a day. In another embodiment, the composition is applied from about once every other day to about three times a day. In yet another embodiment, the composition is applied from about once daily to about twice daily. Once lightening or other desirable effects are achieved, the frequency and dosage can be reduced to a maintenance level. The maintenance level will vary according to the individual, but in one embodiment is from about 1/10 to about ½ of the previous dose and/or frequency. In another embodiment, the maintenance level is from about ⅕ to about ⅓ of the previous dose and/or frequency. The dosages and frequencies listed here are guidelines only and can be modified based on a variety of different factors including the condition of the skin to be treated, the topical or systemic administration of other compounds that might affect the skin, and other systemic conditions such as kidney or liver conditions, that might affect the metabolism of the administered compounds.

The inventive compositions and methods provide effective means of skin treatment. Some conditions that may be treated by these compositions and methods include acne, actinic damage, dandruff, eczema, fine lines, psoriasis, warts and wrinkles. In some cases, particularly in elderly people, these conditions are accompanied by irregularities in pigmentation. Accordingly, certain embodiments of the present invention include skin lighteners for treating such irregular coloration.

The inventive compositions and methods provide effective treatment of the skin without undue systemic exposure to the active ingredients. The inventive compositions and methods are well tolerated, and can be used together with other skin care products and cosmeceuticals. They can be used on a wide variety of individuals and are not likely to provoke allergic or inflammatory reactions due to the increased bioavailability of the drug at lower dosages by virtue of the improved transdermal delivery system.

V. Chemical Denervation Drugs (e.g. BOTOX®) for the Treatment of Rhytides from Muscular Contraction

Wrinkles of the skin are caused either by muscular contraction or by solar-aging. Treatment of wrinkles caused by solar-aging has been treated by collagen bio-synthesis, and is described in more detail above. Muscular contraction convolutes the overlying skin culminating in deep furrows, or wrinkles. Treatment of wrinkles has been effected by temporarily paralyzing the offending muscle.

Clostridia botulina bacteria produce a class of chemical compounds known as toxins. The Botulina Type A toxin is processed and purified to produce a sterile product suitable for specific therapeutic purposes. Once the diluted toxin is injected, it produces a temporary paralysis (chemodenervation) of the muscle by preventing transmission of nerve impulses to the muscle. The duration of the paralysis is generally three to four months. Continuing treatments are necessary to maintain the effects of the toxin over time.

The toxin has been used for a variety of applications, such as for the treatment of strabismus and blepharospasm, or involuntary muscle spasms of the eyelids. The toxin is also used to treat muscle spasms in the face and neck. The toxin (known as BOTOX® and available from Allergan Pharmaceuticals, Inc.) has been approved by the FDA for the treatment of blepharospasm (eyelid spasms), strabismus (crossed eyes), cervical dystonia (spastic muscle disorder of the neck), motor disorders of the facial nerve (cranial nerve VII) and excessive perspiration disorders of the underarms. The FDA has also approved the toxin for the treatment of moderate to severe wrinkling in the glabellar lines, or frown lines, and for the cosmetic treatment of forehead wrinkles caused by specific muscle groups. The toxin has also been used “off-label” to treat other areas of the face and body, such as crows feet wrinkles and neck bands, as well as to treat migraine headaches, colorectal disorders, excessive perspiration disorders of the hand and musculoskeletal pain disorders.

BOTOX® injections are customized for every patient, depending on the individual's needs. These injections can be performed in areas such as the eyelid region, the forehead, and the neck. While BOTOX® cannot prevent aging, it can diminish the appearance of wrinkles caused by the contraction of these muscle groups by paralyzing the muscles, thereby preventing the overlying skin from furrowing as the muscle contracts.

BOTOX® treatments are alternatives to more invasive, surgical treatments. While there are some risks and complications associated with the use of BOTOX®, these risks are minimal, especially when compared with those associated with surgery. Some risks include occasional minor bleeding and bruising, damage to deeper structures during the course of injection, drooping lids and double vision from migration of the toxin into the eyelid, asymmetry of post-injection appearance, and pain at the injection site. Other complications, such as allergic reactions, infection from a contaminated injection site, drug interactions and localized skin reactions, are also possible, but rarely occur.

Prior to injection, the toxin is first reconstituted in 0.9% sterile, non-preserved saline (100 units in 2.5 ml saline). The resulting formulation is will be 4.0 units per 0.1 ml and a total treatment dose of 20 units in 0.5 ml. Although the toxin retains its efficacy up to six weeks after reconstitution in preserved saline, it is generally recommended to store the reconstituted toxin in a refrigerator at 2 to 8° C. and to use the solution within four hours of reconstitution. However, unopened vials may be stored in a refrigerator for up to twenty-four months. The effects of injected BOTOX® generally last for approximately three to four months. More frequent dosing is not recommended. Typical doses for the treatment of glabellar furrows has been 20 to 35 units distributed in 5 to 7 sites.

The other common cause of wrinkles in the skin (rhytides) is enzymatic degradation of the collagenous matrix normally providing support at the epidermal-dermal junction. These rhytides are not caused by muscular contraction. Rather, the loss of this structural integrity occurs as the result of aging (both intrinsic and extrinsic). Gravity plays a role in the formation of these rhytides by allowing the unsupported skin to fall upon itself, culminating in age-related wrinkles. BOTOX® has no effect on these rhytides and skin rejuvenation is required to restore the integrity of the collagenous matrix. Repair or reversal of this damage requires collagen biosynthesis and is discussed in detail above.

Besides BOTOX®, there are two additional botulinium toxin products: Dysport and Reloxin. Dysport is a British product and Reloxin is the U.S. name for Dysport. The dosimetry for Dysport is modified as a result of the availability in 500 unit vials, as opposed to the availability of 100 unit vials of BOTOX®. Approximately 1 unit of BOTOX® is equivalent to approximately 3 units of Dysport. Still other toxins are being developed, such as BTXA (Hugh & Promedic, North China), Estetoxa (Cosmoscience, Beijing, China) and Myobloc (which is actually a type B toxin)(Elan Pharmaceuticals).

According to some embodiments of the present invention, compositions and methods are provided for treatment of the skin with BOTOX® or other chemical denervation drug without the need for injection or other invasive penetration. In one embodiment, for example, a topical composition for the treatment of wrinkles caused by muscular contraction includes a chemical denervation agent, a transdermal delivery agent or composition and a topical pharmaceutically acceptable carrier. The chemical denervation agent may be any such agent capable of temporally denervate or render powerless a target muscle. For example, the chemical denervation agent may be botulinium toxin type A (BOTOX®) or a similar toxin. The transdermal delivery agent or composition may be any suitable penetrant, and in one embodiment, the transdermal delivery agent is as described above. Similarly, the topical pharmaceutically acceptable carrier may be any suitable carrier, and in one embodiment includes the topical pharmaceutically acceptable carrier described above.

According to another embodiment of the present invention, a method of administering the inventive topical composition is provided. According to the method, the topical composition is applied to the target site in the form of a small saturated disc of absorbent material. This prevents unwanted complications such as chemical denervation of adjacent facial or extraocular muscles.

The inventive compositions and methods provide new ways to administer a chemodenervation agent, such as BOTOX®, with increased efficiency and without the pain and discomfort normally associated with penetrating injections. Besides pain and discomfort, injections may cause localized swelling or edema, capillary hemorrhage and inflammation, which are generally avoided when the inventive compositions are used.

The current treatment for wrinkles caused by muscular contraction requires the patient to accept an injection in each target area, such as areas showing frown lines, furrows and/or wrinkles. These injections must be repeated approximately every three to four months to provide a sustained relaxation of the offending muscles and a smoothing of the overlying skin surfaces.

However, the compositions and methods of the present invention include a chemodenervation agent and transdermal delivery agent or composition. The topical compositions may be applied in the form of creams, ointments or saturated absorbent materials (such as cotton pledgets). This enables direct application of the composition over the target site, thereby avoiding inadvertent diffusion into an unwanted area. The inventive compositions and methods also avoid the risks and complications associated with injection, such as localized swelling or edema, capillary hemorrhage and inflammation, as well as pain and discomfort.

### VI. Anti-Fungal Drugs for Treatment of Onychomycosis and Related Ailments

Onychomycosis, dermatomycosis and tinea pedis each refer to a fungal disease with special reference to the site of the inflammatory process. The treatment options for these conditions span the spectrum of debridement, topical therapy and oral therapy.

When the finger or toe nails are involved (onychomycosis), podiatrists commonly use debridement to reduce the thickness of the nails affected by the disease. This approach is reserved primarily for patients experiencing pain and discomfort and are unwilling or unable to take oral anti-fungal medications. Debridement, however, is not curative and should be used in combination with systemic anit-fungals in order to effectively eradicate the onychomycosis.

Current topical onychomycosis treatments alone generally do not cure the condition. However, they may be used when the patient cannot or will not take oral medications. More than 85% of onychomycosis cases are chronic and do not respond to current topical therapies.

In spite of the increasing occurrence of onychomycosis, may patients do not receive adequate treatment. Specifically, 47% of patients do not get treated and remain frustrated with the progress of the disease. Of those receiving treatment, 32% get only mechanical treatment, 7% receive mechanical and topical treatment (prescription or over-the-counter), another 7% received topical therapy only, 5% received only oral anti-fungal treatment and 2% received a combination of oral anti-fungal and mechanical treatment.

Ciclopirox (Penlac™, Dermik Laboratories) nail lacquer is a prescription topical preparation that has less than a 10% cure rate. Treatment can take up to a year. Moreover, debridement is generally recommended in combination with such treatment.

The oral treatment of onychomycosis has been the most efficacious treatment. Oral anti-fungal agents work by penetrating the nail plate from the nail matrix and nail bed. These agents have a “reservoir effect” and the nails retain effective anti-fungal concentrations for months after the treatment has been stopped. Moreover, most patients tolerate the treatment well.

Oral anti-fungal agents include fungicidal terbinafine and fungistatic itraconazole. Relapse rates are low for both agents. While griseofulvin and detoconazole were once the agents of choice, they are now rarely used for the treatment of onychomycosis. Iatraconazole (Sporonax, Janssen Pharmaceuticals) is another oral medication for onychomycosis but is considered fungistatic rather than fungicidal.

Systemic treatment should be used in those with multiple mail involvement or significant involvement of any nail, those with streaks extending to the nail matrix, those who have tried topical treatment without success, and those who are unable to use topical therapy for any reason. The treatment of choice for onychomycosis has been Lamisil (terbinafine, Novartis). Oral terbinafine is generally safe and the adverse side effects relate primarily to gastrointestinal and skin events.

Oral terbinafine therapy generally requires a dosage regimen of 250 mg once daily for several months. Terbinafine is an orally active allylamine. The allylamines are a chemical class of anti-fungal agents. Like azole anti-fungals, terbinafine selectively inhibits the biosynthesis of ergosterol, a component of fungal cell membranes vital to membrane integrity and organism growth. Terbinafine also selectively inhibits production of fungal cell squalene epoxidase, the enzyme that converts squalene to squalene oxide. This action causes a fungicidal accumulation of squalene, which in high concentrations disrupts cell membranes. In vitro, terbinafine is active against Trichophyton mentagrophytes, Trichophyton rubrum, Candida albicans, Epidermphyton floccosum, and Scoplariopsis brevicaulis. Its fungicidal action is primarily against dermatophytes, molds, dimorphic fungi, and the yeast, Candid parapsilosis.

Aside from the possibility of liver failure, the side effects are generally mild. In many applications, it is, however, more desirable to topically apply medications for the treatment of dermatophytic infections. Such an infection is caused by the invasion of fungi into the keratinized layers of the epidermis, hair and nails. While certain anti-fungal agents may be administered topically and orally, topical application has not generally been successful.

The risks associated with oral administration of anti-fungal agents likely could be reduced if the agents could successfully be administered topically. Topical administration has been hindered by the lack of a suitable carrier or transdermal delivery system. Current carrier systems, including highly volatile solvents such as alcohols, and oily solvents or ointments, are ineffective or exhibit other drawbacks. Generally, highly volatile solvents, such as alcohols, dissipate before sufficient time elapses for the anti-fungal to be absorbed through the dermis, leaving a residue on the surface. Furthermore, some carrier solvents that are at least partially effective, including trichloroethanol and dimethylsulfoxide, cause irritation when used over extended periods of time. Accordingly, in one embodiment of the present invention, a topical composition for the treatment of fungal diseases includes a transdermal delivery agent or composition that does not cause irritation or leave a substantial amount of oily residue.

In one embodiment of the present invention, a topical composition is provided for the treatment of onychomycosis, dermatomycosis, tine pedis and other fungal diseases. In one exemplary embodiment, the topical composition includes an appropriate anti-fungal agent, a penetrant or transdermal delivery agent or composition, and a topical pharmaceutically acceptable carrier. The topical composition can take any suitable form, such as a cream, ointment, lotion, etc.

The anti-fungal agent may be any suitable anti-fungal agent. Non-limiting examples of suitable anti-fungal agents include fungicidal agents and fungistatic agents, such as terbinafine, itraconazole, micronazole nitrate, thiapendazole, tolnaftate, clotrimazole and griseofulvin.

The nail apparatus is found only in primates and develops from an in-growth of the epidermis into the dermis. The nail plate is formed by fully keratinized ‘dead’ horn cells (onchocytes). The dermis of the nail apparatus (underlying its epithelial structures) is a fibrocollagenous network lacking subcutaneous tissue and pilosebaceous units. The nail apparatus includes bundles of collagen radiating into the periosteum of distal phalangeal bones, and is situated in a very small space between two hard tissues, the nail plate and the bone. Although the nail apparatus appears simple, it has a rather complex architecture with five distinct anatomical regions including the nail plate as a fully cornified structure, and four highly specialized epithelial tissues: the proximal nail fold, the nail matrix, the nail bed and the hyponychium.

The nail plate can be viewed as equivalent to the epidermal stratum corneum, but shows a very firm attachment to the nail bed and does not desquamate. In contrast to the stratum corneum of the epidermis, which has a fat content of 10%, the total fat content of the nail varies between 0.1 and 5%, with cholesterol as the main lipid constituent. The water content of the normal nail varies between 7 and 18%, and is thus lower than that of the epidermis.

Given these similarities between the nail plate and the stratum corneum, transdermal delivery agents or compositions useful for delivering agents through the stratum corneum can also be used to deliver agents through the nail plate. Accordingly, in one embodiment of the present invention, a transdermal delivery agent for delivering an anti-fungal agent through the nail plate is the same as the transdermal delivery agent described above with respect to delivery through the stratum corneum and epidermis.

The compositions and methods of the present invention provide for effective penetration of the nail plate and dermis of the nail, delivering the anti-fungal agent and effectively eradicating the offending fungal organism. In addition, topical administration of the compositions of the present invention directly to the targeted inflammation avoids the excessive dosimetry and attendant risks and complications normally associated with oral administration of anti-fungal agents. Protracted oral administration of 250 mg daily for as long as six months is currently required to resolve onychomycosis. The cost of this treatment and continued studies to ensure the absence of complications is substantial. Patient compliance is difficult to maintain, and the costs and inconvenience associated with the current treatment methods further deteriorate patient compliance. The inventive compositions and methods, on the other hand, are less costly, require less time for resolution of the disease, and substantially decrease adverse side effects.

### VII. Anesthetics

There are many potential uses for topical anesthetic agents. These uses may include pain relief from burns, contact dermatitides, scrapes, insect stings and bites, pruritus, eczema, sprains, strains, and other soft tissue injuries, dermal wounds, and other conditions affecting the skin or causing a lesser or greater degree of pain or discomfort. Other uses include use as part of or in preparation for surgical procedures or as a pre-treatment for medical penetration wounds, such as those from injection, inoculation or venapuncture.

In general, local anesthetics prevent the generation and conduction of nerve impulses. Their primary site of action is the cell membrane. However, one problem with the topical administration of local anesthetics is their potential systemic toxicity. Therefore, the lowest effective dose of the anesthetic should be used to prevent systemic toxicity. This is complicated by the typically poor penetration of the skin by these anesthetics.

According to some embodiments of the present invention, topical anesthetic compositions are provided which have increased depth of pain relief, and increased speed and duration of relief. In addition, the inventive topical compositions avoid skin irritation and inflammation and significantly improve the skin penetration of the anesthetics. In one embodiment, for example, a topical composition includes at least one anesthetic, a permeation enhancer or transdermal delivery agent or composition, and a topical pharmaceutically acceptable carrier. In one embodiment, the at least one anesthetic includes three local anesthetics, benzocaine, lidocaine and tetracaine. The transdermal delivery agent or composition and the topical pharmaceutically acceptable carrier are as described above. The transdermal delivery of anesthetics according to the present invention is a comfortable, convenient and non-invasive method of administration.

In one exemplary embodiment, a topical composition for the enhanced delivery of local anesthetics includes a local anesthetic or a combination of local anesthetics, a permeation enhancer or transdermal delivery agent or composition, and a pharmaceutically acceptable carrier. The transdermal delivery agent or composition is active a pH ranging from about 3.0 to about 7.4 and the topical composition has a pH within the same range. The transdermal delivery agent or composition is present in the composition in an amount ranging from about 2% to about 20% and may be any suitable penetrant. In one embodiment, the transdermal delivery agent or composition is as described above. Similarly, the pharmaceutically acceptable carrier may be any suitable carrier, and in one embodiment is as described above.

Non-limiting examples of suitable local anesthetics include benzocaine, lidocaine, tetracaine, bupivacaine, cocaine, etidocaine, mepivacaine, pramoxine, prilocalne, procaine, chloroprocaine, oxyprocaine, proparacaine, ropivacaine, dyclonine, dibucaine, propoxycaine, chloroxylenol, cinchocaine, dexivacaine, diamocaine, hexylcaine, levobupivacaine, pyrrocaine, risocaine, rodocaine, pharmaceutically acceptable derivatives and bioisosteres thereof and mixtures thereof.

In one embodiment, the topical composition includes a combination of local anesthetics, for example, a combination of benzocaine, lidocaine and tetracaine. In this combination of anesthetics, benzocaine is present in an amount ranging from about 10% to about 30% by weight. In another embodiment, benzocaine is present in an amount ranging from about 15% to about 25% by weight. In yet another embodiment, benzocaine is present in an amount of about 20% by weight.

In the combination of anesthetics, lidocaine is present in an amount ranging from about 3% to about 12% by weight. In another embodiment, the lidocaine is present in an amount ranging from about 4.5% to about 9% by weight. In yet another embodiment the lidocaine is present in an amount of about 6% by weight.

Finally, the tetracaine is present in an amount ranging from about 2% to about 8% by weight. In an alternative embodiment, the tetracaine is present in an amount ranging from about 3% to about 5% by weight. In yet another embodiment, the tetracaine is present in an amount of about 4% by weight.

According to another embodiment of the present invention, the topical composition may further comprise a an anhydrous delivery system for cooling and drying the skin. The anhydrous delivery system preconditions the skin for penetration and may be used in addition to or in place of the pharmaceutically acceptable carrier.

In one embodiment, the anhydrous delivery system includes a volatile organic co-solvent, menthol, propylene glycol, 2,2′-ethoxyethoxyethanol (diethyleneglycol monoethyl ether), a gelling agent, a preservative and a dispersing agent. The volatile organic co-solvent may include isopropyl alcohol. The anhydrous delivery system provides a pH ranging from about 3.0 to about 7.4 and can further include an acid for adjusting the pH value. Other ingredients may optionally be included in the anhydrous delivery system. Non-limiting examples of these optional ingredients include fragrances, opacifying agents, film forming agents, buffers and vasoconstrictors.

The gelling agent may be hydroxypropylcellulose having a viscosity ranging from about 5 cps to 25,000 cps measured at room temperature. In one embodiment, the hydroxypropylcellulose has a viscosity ranging from about 500 cps to about 5000 cps measured at room temperature. In another embodiment, the hydroxypropylcellulose has a viscosity of about 1500 cps measured at room temperature. The hydroxypropylcellulose may be present in the anhydrous delivery system in a concentration ranging from about 1% to about 2%. Other gelling agents, such as methylcellulose and hydroxypropylmethylcellulose, are known and can be used in place of or in addition to the hydroxypropylcellulose.

The dispersing agent may be glycerin and may be present in the anhydrous delivery system in a concentration of up to about 40%. In one embodiment, the glycerin is present in the anhydrous delivery system in a concentration ranging from about 5% to about 25%.

The preservative is included at a concentration effective to inhibit undesirable effects such as microbial growth, UV light and/or oxygen-induced breakdown of components, and the like. Non-limiting examples of suitable preservatives include butylated hydroxytoluene (BHT) and disodium EDTA. When a preservative is included, it is present at a concentration sufficient to provide a preservative effect. For example, the preservative may be present in a concentration ranging from about 0.01% to about 1.5%. In one embodiment, the preservative is present in an amount ranging from about 0.025% to about 1.0%, depending on the preservative used.

Non-limiting examples of suitable vasoconstrictors include phenylephrine, naphazoline, tetrahydrozoline, oxymetazoline, tramazoline, and salts of these compounds.

According to still another embodiment of the invention, the topical composition may further include a therapeutic agent for augmenting or complementing the anesthetic action and the goal of therapeutic intervention. Non-limiting examples of suitable therapeutic agents include analgesics, antianxiety agents, antiarrhythmics, antibacterials, antibiotics, anticoagulants, anticonvulsants, antifungals, antihistamines, antiinflammatories, antivirals, bronchodilators, calcium channel blockers, cytotoxics, anticancer agents, cytokines, growth factors, immunosuppressives, muscle relaxants, psychotherapeutics, sympathomimetics, vasodilators, vitamins, and other therapeutic agents. For example, a topical anesthetic composition according to one embodiment may contain an anti-itch or antipruritic therapeutic agent. Non-limiting examples of suitable anti-itch agents include antihistamines, for example, alkylamines such as bromphenphiramine maleate, cholorpheniramine maleate and dexchlorpheniramine maleate; ethanolamines such as diphenhydramine HCl, carbinoxamine and clemastine fumarate; ethylenediamines, including pyrilamine maleate; phenothiazines such as promethazine HCl; piperidines such as cyproheptadine HCl; and other antihistamines such as the non-sedating compounds astermazole, loratidine, fexofenadine and cetirizine. Further anti-itch agents include cooling and soothing compounds such as camphor, thymol, calamine and crotamiton.

One exemplary topical composition according to this invention that includes an anesthetic agent and an anti-itch agent includes an alkylamine in an amount ranging from about 0.5% to about 10% based on the total weight of the composition. In one embodiment, for example, the composition contains an alkylamine in an amount ranging from about 0.75% to about 3% based on the total weight of the composition. In yet another embodiment, the composition includes from about 0.5% to about 5% diphenhydramine hydrochloride.

Other active ingredients can also be included. For example, an antibacterial gel may be formulated as above except that the anesthetic is not included and an antibacterial agent is added. Non-limiting examples of suitable antibiotics include aminoglycosides such as streptomycin, neomycin and gentamycin; cepahlosporins such as cephalothin, cefazolin, cefalexin, cefuroxime, cefamandole, cefoxitin and cefaclor; antibiotic glycopeptides such as vancomycin; lincosamides such as clindamycin; macrolides such as erythromycin; nitroimidazoles such as tinidazole; penicillins such as azocillin, nafcillin, methicillin, ampicillin, amoxacillin; sulfonamides; tetracyclines; antibiotic polypeptides such as bacitracin; and quinolones such as ciprofloxacin. Other antibacterials are known and can also be used.

One exemplary antibiotic formulation includes an antibiotic selected from polymyxin B sulfate, bacitracin zinc, neomycin sulfate and combinations thereof. The antibiotic is included in an appropriate dose. For example, polymyxin B sulfate may be included in an amount ranging from about 1000 to about 5000 units per gram of formulation, bacitracin zinc may be included in an amount ranging from about 100 to about 5000 units per gram of formulation, and neomycin sulfate may be included in an amount equivalent to from about 1 to about 25 mg of neomycin base per gram of formulation. In one embodiment, a suitable mixture of antibiotics includes polymyxin B sulfate in an amount of about 1000 units per gram of gel formulation, bacitracin zinc in an amount of about 500 units per gram of gel formulation and neomycin sulfate in an amount equivalent to about 3.5 mg of neomycin base per gram of gel formulation. Other antibiotic formulations, combinations and amounts may be included in a gel formulation as appropriate for the therapeutic application.

Non-limiting examples of suitable dosage forms for the topical administration of the inventive topical compositions include creams, gels, ointments and topical sprays. The active components are admixed with a physiologically acceptable carrier and any preservatives, buffers, or propellants as may be required. In certain embodiments, ophthalmic formulations, eye ointments, powders and solutions, as well as dental formulations containing appropriate flavors and sweeteners are provided. The topical anesthetic compositions according to the present invention can be packaged in spray bottles or other suitable delivery devices, and can be applied to the surface of the skin utilizing a cotton swab, gauze pad, or other suitable applicator.

In yet another embodiment of the invention, a method for enhancing the flux of a local anesthetic through a bodily surface includes administering the anesthetic to a localized region of the body and administering a permeation enhancer or transdermal delivery agent or composition to the localized region during administration of the anesthetic. The permeation enhancer may have a pH ranging from about 3.0 to about 7.4. The local anesthetic may be administered before or after the administration of the permeation enhancer. Alternatively, the local anesthetic may be administered simultaneously with the permeation enhancer. For example, the anesthetic(s) and permeation enhancer may be included in a single topical composition which can include additional ingredients, such as a topical pharmaceutically acceptable carrier.

According to still yet another embodiment, a system for the topical administration of a local anesthetic includes at least one drug reservoir containing a local anesthetic and a permeation enhancer or transdermal delivery agent or composition, and means for maintaining an interface between the at least one drug reservoir and a surface of the body. Non-limiting examples of suitable drug reservoirs include sponges, pads, patches, polymer matrices, bandages, swabs and any other device capable of holding a sufficient quantity of the local anesthetic(s). The drug reservoir may have any suitable size and shape or application to the intended site on the body. For example, the drug reservoir may be square, rectangular, circular, ovular, rectangular, polygonal, etc.

Non-limiting examples of the means for maintaining an interface between the drug reservoir and a surface of the body include adhesives capable of holding the drug reservoir against the surface of the body sufficiently tightly to enable the continued administration of the anesthetic(s) in the drug reservoir to the surface of the body. Various types of adhesives may be used, non-limiting examples of which include water/lipid emulsions, hot melt pressure sensitive adhesives (PSAs), solvent based PSAs, silicone based PSAs, resin emulsion adhesives, water based adhesives, polyacrylate adhesives, rubber adhesives, polystyrene-polybutadiene-polystyrene adhesives, polystyrene-polyisoprene-polystyrene adhesives, polystyrene-poly(ethylene-butylene)-polystyrene block polymer adhesives, vinyl acetate resin adhesives, acrylic ester copolymer adhesives, vinyl acetate/diocyl maleate copolymer adhesives, acrylic copolymer adhesives, and any combination thereof.

In one embodiment, for example, the adhesive may be a pressure sensitive adhesives (PSAs). The PSA may further include a polymer and a humectant. Non-limiting examples of suitable polymers include starches, starch derivatives, vinyl acetate copolymers, polyvinyl pyrrolidone, polyethylene oxide, algin, derivatives of algin, polyacrylate quats, polymaleic acid, polymaleic anhydride, polyurethanes, polyureas, karaya, gum acacia, locust bean gum, xanthan gum, guar gum, modified guar gum, maltodextrin, carboxymethyl cellulose, carboxypropyl cellulose, polyacrylamide, polyvinyl alcohol, poly AMPS (poly(2-acrylamido-2-methylpropanesulfonic acid)), polyacrylates, and combinations thereof. Non-limiting examples of suitable humectants include glycerin and polyhydric acids such as ethylene glycol, propylene glycol, triethylene glycol, tetraethylene glycol, and sorbitol. The PSA can further include water.

The adhesive can be located on any portion of the drug reservoir. In one embodiment, the adhesive is located on substantially the entire surface of the reservoir that contacts the surface of the body. When the drug reservoir is a patch, the patch can take any suitable shape, such as square, rectangular, circular, ovular, polygonal, etc., and can include a backing. The backing has a front side (the side exposed to the skin during use) and a back side (the side exposed to the environment during use). In such an embodiment, the adhesive as well as the anesthetic and permeation enhancer are located on the front side of the backing. The backing can include a porous sheet of water insoluble material that provides support for the patch. The backing should be non-irritating to the skin. Optionally, the backing is breathable and/or vapor permeable. The backing may also be porous since porosity provides opening from receiving the local anesthetic and permeation enhancer. Porosity also impart vapor permeability. The backing may be woven or non-woven and can be made of any suitable material that is capable of forming a flexible, bendable, pliable and/or stretchable sheet of water insoluble porous material.

In one embodiment, the patch, upon contact with the skin, allows the skin to breathe. According to another embodiment, the patch, upon prolonged contact with the skin, holds the anesthetic and permeation enhancer in place while allowing the skin to breathe over extended periods of time, e.g. up to about 10 days. In one embodiment, the patch contacts the skin for about 1 day. In another embodiment, the patch contacts the skin for about 8 hours. Because the patch is in contact with the skin for extended periods of time, the patch may be vapor permeable and non-occlusive such that the skin is able to breathe.

The backing of the patch may be a porous, self-supporting sheet of water insoluble, polymeric or natural material that provides strength and integrity for the drug reservoir. For example, the backing may include water insoluble polymeric fibers, open cell foam (e.g. polyurethane, polyvinyl chloride or polyethylene), a porous film or any other kind of matrix with spaces within the matrix. In one embodiment, the backing is selected from polyester, polyurethane, polyolefin, polyamide fibers, natural fibers, cotton fibers, polycellulose fibers and mixtures thereof.

One exemplary backing suitable for use in the present invention is a lightweight, porous, pliable strip of nonwoven fabric of polymeric or natural fibers such as polyester, cotton or cellulose fibers. Additional, stable, water insoluble sheet materials are known and can also be used. The coating of the local anesthetic and permeation enhancer onto or into the backing may be accomplished using a continuous process mixer.

In one embodiment, the adhesive on the patch is attached to a removable backing liner. The backing liner helps maintain the adhesive properties of the patch prior to use, such as during manufacturing, packaging, shipping and/or storage. Backing liners are well known, and any suitable backing liner may be used. The backing liner may be provided with a tab section and may include a perforation allowing the tap section of the backing liner to be removed. Removal of the tab section allows the patch to be removed from the backing liner with ease.

The following Example is presented for illustrative purposes only, and is not intended to limit the scope of the invention.

### Example 1

### Preparation of a Gel Having Benzocaine, Lidocaine, Tetracaine and PLO

6.0 g of benzocaine, 1.8 g of lidocaine and 1.2 g of tetracaine were weighed and 2 ml dimethylsulfoxide, 3 ml benzyl alcohol, 7 ml of lecithin-isopropyl palmitate and 6 ml of 69% ethanol were added thereto and the ingredients mixed. 18 ml of Pluronic F12730% gel was added and the composition was milled once at a #1 setting. The study discussed above regarding transdermal penetration of the inventive transdermal delivery compositions was performed using the resulting compositions, and the results were the same as reported above.

The inventive compositions, devices and methods provide a way to administer local anesthetics with increased efficiency. According to the inventive, compositions, devices and methods, lower doses of the anesthetic can be used to achieve the same degree of anesthesia, thereby reducing the occurrence of side effects such as irritation, inflammation and hypersensitivity. The inventive compositions, devices and methods are highly effective in increasing the depth, speed and duration of pain relief. Moreover, the inventive compositions, devices and methods reduce systemic toxicity.

### VIII. Non-Steroidal Anti-Inflammatories

Prostaglandins are a related family of chemicals that are produced by the cells of the body and have several important functions. They promote inflammation, pain and fever, support the function of platelets that are necessary for the clotting of blood, and protect the lining of the stomach from the damaging effects of acid. Prostaglandins are produced within the body's cells by the enzyme cyclooxygenase (Cox). There are actually two Cox enzymes, Cox-1 and Cox-2. Both enzymes produce prostaglandins that promote inflammation, pain and fever. However, only Cox-1 produces prostaglandins that support platelets and protect the stomach. Non-steroidal anti-inflammatory drugs (NSAIDs) block the Cox enzymes and reduce prostaglandins throughout the body. As a consequence, ongoing inflammation, pain and fever are reduced. Since the prostaglandins that protect the stomach and support the platelets and blood clotting are also reduced, NSAIDs can cause ulcers in the stomach and promote bleeding. NSAIDs differ in how strongly they inhibit Cox-1 and therefore differ in their tendency to cause ulcers and promote bleeding.

NSAIDs are used primarily to treat inflammation, mild to moderate pain, and fever. Specific uses include the treatment of headaches, arthritis, sports injuries and menstrual cramps. Aspirin is an NSAID used to inhibit the clotting of blood and prevent strokes and heart attacks in individuals at high risk. NSAIDs are also included in many cold and allergy preparations.

NSAIDs vary in their potency, duration of action, and the way in which they are expelled from the body. They also differ in their tendency to cause ulcers and promote bleeding. the more an NSAID blocks Cox-1, the greater its tendency to cause ulcers and promote bleeding. One NSAID, celecoxib (CELEBREX®), blocks Cox-2, but has little effect on Cox-1. This drug is referred to as a selective Cox-2 inhibitor and causes less bleeding and fewer ulcers than other NSAIDs. Aspirin is a unique NSAID, not only because of its many uses, but also because it is the only NSAID capable of inhibiting blood clotting for a prolonged period of time (e.g. 4 to 7 days). This prolonged effect makes aspirin an ideal drug for preventing blood clots that cause heart attacks and strokes. Most other NSAIDs inhibit blood clotting for only a few hours. Ketorolac usually requires narcotics and causes ulcers more frequently than any other NSAID. Ketorolac is therefore not used for more than five days. Although NSAIDs have a similar mechanism of action, individuals who do not respond to one NSAID may respond to another.

NSAIDs are associated with a number of side effects. The frequency of side effects varies between the different drugs, but the most common side effects include nausea, vomiting, diarrhea, constipation, decreased appetite, rash, dizziness, headache and drowsiness. NSAIDs may also cause fluid retention, leading to edema. the most serious side effects are kidney failure, liver failure, ulcers and prolonged bleeding after an injury or surgery. Some individuals are allergic to NSAIDs and may develop shortness of breath when and NSAID is administered. People with asthma are at higher risk of experiencing serious allergic reactions to NSAIDs. Individuals with serious allergies to an NSAID are likely to experience a similar reaction to different NSAIDs. Use of aspirin in children and teenagers with chicken pox or influenza has been associated with the development of Reye's syndrome. Therefore, aspirin and non-aspirin salicylates (e.g. salsalate) should not be used in children and teenagers suspected of having or having chicken pox or influenza.

NSAIDs reduce blood flow to the kidneys and therefore reduce the action of diuretics and decrease the elimination of lithium (Eskalith) and methotrexate (Rheumatrex). NSAIDs also decrease the ability of the blood to clot and therefore increase bleeding time. When used with other drugs that also increase bleeding time, there in an increased likelihood of bleeding complications. Therefore, individuals taking drugs that reduce the ability of blood to clot should avoid prolonged use of NSAIDs. NSAIDs may also increase blood pressure in patients with hypertension and therefore antagonize the action of drugs that are used to treat hypertension.

The complete list of approved NSAIDs is very long, but the following are some of the most commonly used: aspirin, salsalate (Amigesic), diflunical (Dolobid), ibuprofen (Motrin), ketoprofen (Orudis), nabumetone (Relafen), piroxicam (Feldene), naproxen (Aleve, Naprosyn), diclofenac (Voltaren), indomethacin (Indocin), sulindac (Clinoril), tolmetin (Tolectin), etodolac (Lodine), ketorlac (Toradol), oxaprozin (Daypro), and celecoxib (Celebrex).

In one embodiment of the present invention, a composition for the topical administration of NSAIDs is provided. The composition may include a NSAID, a permeation enhancer or transdermal delivery agent or composition and a pharmaceutically acceptable carrier. The topical composition has a pH ranging from about 3.0 to about 7.4. The permeation enhancer is active at a pH ranging from about 3.0 to about 7.4 and may be any suitable penetration enhancer. In one embodiment, the penetration enhancer is as described above. Similarly, the pharmaceutically acceptable carrier may be any suitable carrier, and in one embodiment is as described above.

The NSAID may be a single NSAID or a combination of NSAIDs. Non-limiting examples of suitable NSAIDs include aspirin, salsalate, diflunical, ibuprofen, ketoprofen, nabumetone, piroxicam, naproxen, diclofenac, indomethacin, sulindac, tolmetin, etodolac, ketorolac, oxaprozin, celecoxib, and pharmaceutically acceptable derivatives thereof.

In an alternative embodiment, the topical composition may further include an anhydrous delivery system. The anhydrous delivery system is the same as that described above with respect to topical anesthetic compositions and may include a volatile organic co-solvent, menthol, propylene glycol, 2,2′-ethoxyethoxyethanol (diethyleneglycol monoethyl ether), a gelling agent, a preservative, and a dispersing. These components are the same as described above with respect to the anydrous delivery system in the topical anesthetic composition.

The inventive compositions may be applied to the target site in the form of small drug-saturated discs of absorbent material, or in the form of creams, lotions, ointments and like. Such application procedures ensure that the drug is delivered locally to the target site and avoids unwanted diffusion of the drug into other adjacent areas.

According to the inventive compositions and methods for the topical administration of NSAIDs, local soft tissue and joint concentrations are increased while systemic distribution of the drug is decreased, thereby reducing associated side effects. Some side effects are dose-related, however effective transdermal delivery according to certain embodiments of the invention result in increased bioavailability and bioactivity with lower doses. In addition, the compositions and methods according to the present invention reduce or eliminate gastrointestinal complications, such as nausea, vomiting, diarrhea, constipation and decreased appetite usually associated with oral administration of NSAIDs.

Certain exemplary embodiments of the present invention have been illustrated and described. However, those of ordinary skill in the art will understand that various modifications and alternations to the described embodiments may be made without departing from the principal, spirit and scope of the invention, as defined in the appended claims. For example, it is understood that any methods of topical application, administration or treatment described with respect to one topical composition may generally be used with any other topical composition. In addition, it is understood that any pharmaceutically acceptable carrier, anydrous delivery system and transdermal delivery agent or composition may be used with any topical composition, and are not limited to use in the topical compositions where they are initially described. Also, it is understood that any topical composition may be used in conjunction with any non-ablative, mechanical and/or radiation-based therapy as part of the treatment procedure.

What is claimed is:

1. A transdermal delivery composition for topical application on skin, the transdermal delivery composition comprising first and second penetrants, the first and second penetrants working synergistically and following disparate biochemical pathways, wherein the composition has a pH ranging from about 3.0 to about 7.4.

2. The transdermal delivery composition according to claim 1, wherein each of the first and second penetrants is independently selected from the group consisting of:
lower alkyl diols,
C₁₀-C₂₀ fatty acids and esters thereof,
C₄-C₂₀ substituted aliphatic alcohols,
C₄-C₂₀ unsubstituted aliphatic alcohols,
lecithin organogel in isopropyl palrnitate organic solvent,
pluronic lecithin organogel in isopropyl palmitate organic solvent,
a formulation comprising Fe (iron) and Ca (calcium) peptide,
a mixture of 1-dodecylazacycloheptan-2-one with a diol compound or a second N-substituted alkyl-azacycloalkyl-2-one (a “cycloketo” compound), wherein the diol compound is selected from the group consisting of 1,2-propanediol, 1,3-propanediol, 1,2-butanediol, 1,3-butanediol, 1,4-butanediol, and 2,3-butanediol,
aminopolysaccharides,
diisopropyl adipate,
dimethyl isosorbide,
propylene glycol,
1,2,6-hexanetriol,
dioctyl maleate,
propylene carbonate, and
diisopropyl sebacate.

3. The transdermal delivery composition according to claim 1, wherein the first penetrant comprises benzyl alcohol.

4. The transdermal delivery composition according to claim 3, wherein the benzyl alcohol is present in an amount ranging from about 1% to about 20% by weight.

5. The transdermal delivery composition according to claim 3, wherein the benzyl alcohol is present in an amount ranging from about 1.5% to about 2.5% by weight.

6. The transdermal delivery composition according to claim 1, wherein the second penetrant comprises a lecithin organogel.

7. The transdermal delivery composition according to claim 6, wherein the lecithin organogel is pluronic lecithin organogel.

8. The transdermal delivery composition according to claim 6, wherein the lecithin organogel is present in an amount ranging from about 0.5% to about 20% by weight.

9. The transdermal delivery composition according to claim 6, wherein the lecithin organogel is present in an amount ranging from about 0.5% to about 0.6% by weight.

10. The transdermal delivery composition according to claim 1, wherein the first penetrant is benzyl alcohol and the second penetrant is lecithin organogel.

11. The transdermal delivery composition according to claim 1, further comprising at least two metallic cations.

12. The transdermal delivery composition according to claim 11, wherein the at least two metallic cations are selected from the group consisting of Fe, Ca and Cu.

13. The transdermal delivery composition according to claim 11, wherein the at least two metallic cations comprise metal cation peptides.

14. A transdermal delivery composition for topical application to skin, the transdermal delivery composition comprising benzyl alcohol and lecithin organogel, wherein the benzyl alcohol is present in an amount greater than an amount of the lecithin organogel.

15. The transdermal delivery composition according to claim 14, wherein the transdermal delivery composition has a pH ranging from about 3.0 to about 7.4.

16. The transdermal delivery composition according to claim 14, wherein the benzyl alcohol is present in an amount of about 2% by weight and the lecithin organogel is present in an amount of about 0.6% by weight.

17. A topical composition for topical application to skin, the topical composition comprising:
the transdermal delivery composition according to claim 1;
an active ingredient selected from the group consisting of drugs, agents or compositions; and
a topical pharmaceutically acceptable carrier.

18. The topical composition according to claim 17, wherein the topical pharmaceutically acceptable carrier comprises:
dimethyl sulfoxide;
lecithin;
ethanol;
an isopropyl ester of a long-chain fatty acid selected from the group consisting of isopropyl palmitate, isopropyl stearate and isopropyl myristate; and
a nonionic surfactant comprising at least one free hydroxyl group.

19. The topical composition according to claim 17, wherein the topical pharmaceutically acceptable carrier comprises:
water;
propylene glycol;
carbopol;
an octyl ester of a long-chain fatty aid selected from the group consisting of octyl palmitate, octyl stearate, and octyl myristate;
silicone fluid;
cetearyl alcohol;
a buffer for buffering the pH of the composition to a value ranging from about 3.0 to about 7.4; and
at least one non-sensitizing preservative.

20. The topical composition according to claim 17, wherein the topical pharmaceutically acceptable carrier comprises:
propylene glycol;
carbopol;
a surface coated starch polymer;
octyl palmitate;
isopropyl palmitate;
silicone fluid;
glyceryl stearate or PEG-100 stearate;
cetearyl alcohol;
stearic acid;
triethanolamine;
caprylic or capric triglyceride;
caprylic or capric stearyl triglyceride; and
water.

21. The topical composition according to claim 17, wherein the active ingredient comprises a composition comprising:
methionine;
cysteine;
a mixture of amino acids comprising leucine, lysine, phenylalanine, threonine, tryptophan, valine, histidine and arginine;
at least one antioxidant;
at least one cross-linking agent; and
at least one metallic catalyst.

22. The topical composition according to claim 21, wherein the methionine is present in the active ingredient composition in an amount ranging from about 0.0005% to about 0.02% by weight.

23. The topical composition according to claim 21, wherein the cysteine is present in the active ingredient composition in an amount ranging from about 0.01% to about 0.4% by weight.

24. The topical composition according to claim 21, wherein the mixture of amino acids comprises from about 0.005% to about 0.5% by weight of the active ingredient composition.

25. The topical composition according to claim 21, wherein the at least one antioxidant is selected from the group consisting of lipoic acid, lipoic acid derivatives or analogues, ascorbic acid, and ascorbic acid derivatives.

26. The topical composition according to claim 25, wherein the at least one antioxidant is selected from the group consisting of dihydrolipoic acid, lipoic acid esters, dihydrolipoic acid esters, lipoic acid amides, dihydrolipoic acid amides, salts of lipoic acid and salts of dihydrolipoic acid.

27. The topical composition according to claim 25, wherein the at least one antioxidant is α-lipoic acid.

28. The topical composition according to claim 27, wherein the α-lipoic acid is present in the active ingredient composition in an amount ranging from about 0.3% to about 2.0% by weight.

29. The topical composition according to claim 25, wherein the at least one antioxidant is selected from the group consisting of ascorbyl palmitate, ascorbyl myristate, ascorbyl stearate, and ascorbyl isotetrapalmitate.

30. The topical composition according to claim 29, wherein the at least one antioxidant is ascorbyl isotetrapalmitate, and the ascorbyl isotetrapalmitate is present in the active ingredient composition in an amount ranging from about 0.1% to about 0.6% by weight.

31. The topical composition according to claim 25, wherein the at least one antioxidant is a derivative of ginkgo selected from the group consisting of ginkgolide A, ginkgolide B, ginkgolide C and bilobalide.

32. The topical composition according to claim 25, wherein the at least one antioxidant is an isoflavone selected from the group consisting of genistein, genistin, 6″-0-malonylgenistin, 6″-0-acetylgenistin, daidzein, daidzin, 6″-0-malonyldaidzin, 6″-0-acetylgenistin, glycitein, glycitin, 6″-0-malonylglycitin, and 6-0-acetylglycitin.

33. The topical composition according to claim 21, wherein the metallic catalyst is copper in a form selected from the group consisting of cuprous ionic forms, cupric ionic forms, peptide forms, and salt forms selected from the group consisting of cupric acetate, cuprous acetate, cuprous chloride, cupric chloride, cuprous sulfate, and cupric sulfate.

34. The topical composition according to claim 21, wherein the mixture of essential amino acids comprises:
from about 5% to about 20% of leucine;
from about 10% to about 25% of lysine;
from about 5% to about 20% of phenylalanine;
from about 5% to about 25% of threonine;
from about 5% to about 20% of tryptophan;
from about 10% to about 25% of valine;
from about 5% to about 20% of histidine; and
from about 5% to about 20% of arginine.

35. The topical composition of claim 21, wherein the cross-linking agent is a bioflavonoid selected from the group consisting of quercetin, quercitrin, kaempferol, kaempferol 3-rutinoside, 3′-methoxy kaempferol 3-rutinoside, 5,8,4′-trihydroxyl-6,7-dimethoxyflavone, catechin, epicachetin, epicachetin gallate, epigallocachetin gallate, hesperidin, naringin, rutin, vixetin, proanthocyanidin, apigenin, myricetin, tricetin, quercetin, naringin, kaempferol, luteolin, biflavonyl, silybin, silydianin, and silychristin.

36. The topical composition according to claim 35, wherein the bioflavonoid is selected from the group consisting of proanthocyanidin and silybin, and the bioflavonoid is present in an amount ranging from about 0.3% to about 2.0% by weight.

37. The topical composition according to claim 21 wherein the cross-linking agent is decorin.

38. The topical composition according to claim 21, wherein the active ingredient composition further comprises a chaotropic agent.

39. The topical composition according to claim 38, wherein the chaotropic agent is Ca(OH)₂.

40. The topical composition according to claim 21, wherein the active ingredient composition further comprises a long-chain fatty acid ester of tocopherol selected from the group consisting of tocopheryl palmitate, tocopheryl myristate, and tocopheryl stearate.

41. The topical composition according to claim 17, wherein the active ingredient comprises a skin lightener selected from the group consisting of hydroquinone, kojic acid, azelaic acid, glycolic acid and artocarpin.

42. The topical composition according to claim 41, wherein the skin lightener comprises hydroquinone, and the hydroquinone is present in an amount ranging from about 1.5% to about 4.0% by weight.

43. The topical composition according to claim 17, wherein the active ingredient comprises a retinoid selected from the group consisting of isotretinoin, retinal, retinol, retinoic acid, retinyl acetate, retinyl palmitate, retinyl propionate, synthetic retinoid mimics and tretinoin.

44. The topical composition according to claim 43, wherein the active ingredient is tretinoin and the tretinoin is present in an amount ranging from about 0.005% to about 1.0% by weight.

45. The topical composition according to claim 17, wherein the active ingredient comprises:
a retinoid selected from the group consisting of isotretinoin, retinal, retinol, retinoic acid, retinyl acetate, retinyl palmitate, retinyl propionate, synthetic retinoid mimics and tretinoin; and
a skin lightener selected from the group consisting of hydroquinone, kojic acid, azelaiz acid, glycolic acid and artocarpin.

46. The topical composition according to claim 45, wherein the retinoid is tretinoin and is present in an amount ranging from about 0.005% to about 1.0% by weight, and wherein the skin lightener is hydroquinone and is present in an amount ranging from about 1.5% to about 4.0% by weight.

47. The topical composition according to claim 17, wherein the active ingredient comprises a chemical denervation agent.

48. The topical composition according to claim 47, wherein the chemical denervation agent is selected from the group consisting of botulinium type A toxins and botulinium type B toxins.

49. The topical composition according to claim 17, wherein the active ingredient is an anti-fungal agent.

50. The topical composition according to claim 49, wherein the anti-fungal agent is selected from the group consisting of fungicidal agents and fungistatic agents.

51. The topical composition according to claim 50, wherein the anti-fungal agent is selected from the group consisting of terbinafine, itraconazole, micronazole nitrate, thiapendazole, tolnaftate, clotrimazole and griseofulvin.

52. The topical composition according to claim 49, wherein the anti-fungal is present in an amount ranging from about 0.05% to about 3% by weight.

53. The topical composition according to claim 17, wherein the active ingredient is at least one local anesthetic.

54. The topical composition according to claim 53, wherein the at least one local anesthetic is selected from the group consisting of benzocaine, lidocaine, tetracaine, bupivacaine, cocaine, etidocaine, mepivacaine, pramoxine, prilocalne, procaine, chloroprocaine, oxyprocaine, proparacaine, ropivacaine, dyclonine, dibucaine, propxycaine, choroxylenol, cinchocaine, dexivacaine, diamocaine, hexylcaine, levobupivacaine, pyrrocaine, risocaine, rodocaine, pharmaceutically acceptable derivatives and bioisoteres thereof, and mixtures thereof.

55. The topical composition according to claim 53, wherein the at least one anesthetic comprises benzocaine, lidocaine and tetracaine.

56. The topical composition according to claim 55, wherein the benzocaine is present in an amount ranging from about 10% to about 30% by weight, the lidocaine is present in an amount ranging from about 3% to about 12% by weight, and the tetracaine is present in an amount ranging from about 2% to about 8% by weight.

57. The topical composition according to claim 53, further comprising a therapeutic agent selected from the group consisting of analgesics, antianxiety agents, antiarryhthmics, antibacterials, antibiotics, anticoagulants, anticonvulsants, antifungals, antihistamines, antiinflammatories, antivirals, antipruritics, bronchodilators, calcium channel blockers, cytotoxic agents, anticancer agents, cytokines, growth factors, immunosuppressants, muscle relaxants, psychotherapeutics, sympathomimetics, vasodilators, and vitamins.

58. The topical composition according to claim 57, wherein the therapeutic agent is an antihistamine selected from the group consisting of bromphenphiramine maleate, chlorpheniramine maleate, dexchlorpheniramine maleate, diphenhydramine hydrochloride, carbinoxamine, clemastine fumarate, pyrilamine maleate, promethazine hydrochloride, cyproheptadine hydrochloride, astemazole, loratidine, fexofenadine and cetirizine.

59. The topical composition according to claim 57, wherein the therapeutic agent is an antibiotic selected from the group consisting of streptomycin, neomycin, gentamycin, cephalothin, cefazolin, cefalexin, cefuroxime, cefamandole, cefoxitin, cefaclor, vancomycin, clindamycin, erythromycin, tinidazole, penicillin, azocillin, nafcillin, methicillin, ampicillin, amoxicillin, sulfonamides, tetracyclines, bacitracin, polymyxin and ciprofloxacin.

60. The topical composition according to claim 17, wherein the active ingredient is a non-steroidal anti-inflammatory drug.

61. The topical composition according to claim 60, wherein the non-steroidal anti-inflammatory drug is selected from the group consisting of aspirin, salsalate, diflunisal, ibuprofen, ketoprofen, nabumetone, piroxicam, naproxen, diclofenac, indomethacin, sulindac, tolmetin, etodolac, ketorolac, oxaprozin, and celecoxib.

62. The topical composition according to claim 60, wherein the non-steroidal anti-inflammatory drug is present in an amount ranging from about 0.1% to about 80% by weight.

63. A topical composition for topical application to skin, the topical composition comprising:
an active ingredient selected from the group consisting of drugs, agents or compositions;
the transdermal delivery composition according to claim 1; and
an anhydrous delivery system.

64. The topical composition according to claim 63, wherein the anhydrous delivery system comprises:
a volatile organic co-solvent;
menthol;
propylene glycol;
2,2′-ethoxyethoxyethanol;
a gelling agent;
a preservative; and
a dispersing agent.

65. The topical composition according to claim 64, wherein the volatile organic co-solvent is isopropyl alcohol.

66. The topical composition according to claim 64, wherein the gelling agent is selected from the group consisting of hydroxypropylcellulose, methylcellulose, and hydroxypropylmethylcellulose.

67. The topical composition according to claim 64, wherein the dispersing agent is glycerin.

68. The topical composition according to claim 64, wherein the preservative is selected from the group consisting of butylated hydroxytoluene and EDTA.

69. The topical composition according to claim 64, wherein the anhydrous delivery system further comprises a fragrance.

70. The topical composition according to claim 64, wherein the anhydrous delivery system further comprises a vasoconstrictor selected from the group consisting of phenylephrine, naphazoline, tetrahydrozoline, oxymetazoline, tramazoline, and salts thereof.

71. A method of treating skin comprising:
applying the transdermal delivery composition of claim 1 to the skin; and
applying an active ingredient to the skin, the active ingredient being selected from the group consisting of drugs, agents or compositions.

72. The method according to claim 71, wherein the transdermal delivery composition is applied to the skin before, after or simultaneously with application of the active ingredient.

73. The method according to claim 71, further comprising treating the skin with a non-ablative modality.

74. The method according to claim 73, wherein the non-ablative modality is selected from the group consisting of mechanical and radiation-based therapies.

75. The method according to claim 73, wherein the non-ablative modality is selected from the group consisting of microdermabrasion, laser based skin remodeling and radio-frequency-based skin remodeling.

76. The method according to claim 73, wherein the skin is treated with the active ingredient before, after or before and after the skin is treated with the non-ablative treatment modality.

77. The method according to claim 71, wherein the active ingredient comprises an active ingredient composition comprising:
methionine;
cysteine;
a mixture of amino acids comprising leucine, lysine, phenylalanine, threonine, tryptophan, valine, histidine and arginine;
at least one antioxidant;
at least one cross-linking agent; and
at least one metallic catalyst.

78. The method according to claim 71, wherein the active ingredient comprises a retinoid.

79. The method according to claim 71, wherein the active ingredient comprises a skin lightener.

80. The method according to claim 71, wherein the active ingredient comprises a retinoid and a skin lightener.

81. The method according to claim 71, wherein the active ingredient comprises a chemical denervation agent.

82. The method according to claim 71, wherein the active ingredient comprises an anti-fungal agent.

83. The method according to claim 71, wherein the active ingredient comprises at least one local anesthetic.

84. The method according to claim 71, wherein the active ingredient comprises a non-steroidal anti-inflammatory drug.

85. A method of treating skin comprising:
applying the transdermal delivery composition of claim 1 to the skin;
applying an active ingredient to the skin, the active ingredient being selected from the group consisting of drugs, agents or compositions; and
treating the skin with a non-ablative treatment modality.

86. The method according to claim 85, wherein the non-ablative treatment modality is selected from the group consisting of mechanical and radiation-based therapies.

87. The method according to claim 85, wherein the non-ablative treatment modality is selected from the group consisting of microdermabrasion, laser-based skin remodeling and radio-frequency based skin remodeling.

88. The method according to claim 85, wherein the skin is treated with a non-ablative treatment modality before, after or before and after the skin is treated with the active ingredient.

89. A method of rejuvenating and repairing damage to skin, the method comprising topically applying the composition according to claim 21 to the skin.

90. A method of promoting collagen biosynthesis in skin, the method comprising topically applying the composition according to claim 21 to the skin.

91. A method of treating hyperpigmentation of skin, the method comprising topically applying the topical composition according to claim 41 to the skin.

92. A method of treating a skin condition selected from the group consisting of acne, actinic damage, dandruff, eczema, fine lines, psoriasis, warts and wrinkles, the method comprising topically applying the topical composition according to claim 44 to the skin.

93. A method of treating a skin condition selected from the group consisting of acne, actinic damage, dandruff, eczema, fine lines, psoriasis, warts and wrinkles, the method comprising topically applying the topical composition according to claim 46 to the skin.

94. A method of increasing permeation of a chemical denervation agent through skin, the method comprising topically applying the topical composition according to claim 48 to the skin.

95. A method of treating rhytides caused by muscular contraction, the method comprising topically applying the topical composition according to claim 47 to the skin.

96. A method of treating a fungal infection comprising topically applying the topical composition according to claim 49 to an affected area.

97. A method of treating onychomycosis comprising topically applying the topical composition according to claim 49 to an affected area.

98. A method of administering a local anesthetic through skin, the method comprising topically administering the topical composition according to claim 53 to the skin.

99. A method of administering a non-steroidal anti-inflammatory drug through skin, the method comprising topically applying the topical composition according to claim 60 to the skin.

100. A system for administering an active ingredient through skin, the system comprising:
a drug reservoir comprising the active ingredient and the transdermal delivery composition according to claim 1; and
means for sustaining contact between the drug reservoir and the skin.

101. The system according to claim 100, wherein the drug reservoir is selected from the group consisting of sponges, pads, patches, polymer matrices, bandages, and swabs.

102. The system according to claim 100, wherein the means for sustaining contact between the drug reservoir and the skin is an adhesive.

103. A topical composition for promotion of collagen synthesis, the composition comprising:
methionine;
cysteine;
a mixture of amino acids comprising leucine, lysine, phenylalanine, threonine, tryptophan, valine, histidine and arginine;
at least one antioxidant;
at least one cross-linking agent; and
at least one metallic catalyst.

104. The topical composition according to claim 103, wherein the at least one antioxidant is selected from the group consisting of lipoic acid, lipoic acid derivatives or analogues, ascorbic acid, and ascorbic acid derivatives.

105. The topical composition of claim 103, wherein the cross-linking agent is a bioflavonoid selected from the group consisting of quercetin, quercitrin, kaempferol, kaempferol 3-rutinoside, 3′-methoxy kaempferol 3-rutinoside, 5,8,4′-trihydroxyl-6,7-dimethoxyflavone, catechin, epicachetin, epicachetin gallate, epigallocachetin gallate,

106. The topical composition according to claim 103, wherein the cross-linking agent is decorin.

107. A method of treating skin, the method comprising:
applying the composition according to claim 103 to the skin; and
treating the skin with a non-ablative treatment modality.

108. The method according to claim 89, wherein the non-ablative treatment modality is selected from the group consisting of mechanical and radiation-based therapies.

109. The method according to claim 103, wherein the non-ablative treatment modality is selected from the group consisting of microdermabrasion, laser-based skin remodeling and radio-frequency based skin remodeling.

110. The method according to claim 103, further comprising applying a transdermal delivery agent to the skin.

111. The method according to claim 110, wherein the transdermal delivery agent is applied to skin before, after or simultaneously with the composition according to claim 103

112. The method according to claim 110, wherein the transdermal delivery agent comprises the composition according to claim 1.
